(19) United States
(12) Patent Application Publication
(10) Pub. No.: US 2015/0157383 A1
(43) Pub. Date: Jun. 11, 2015

(54) ULTRASONIC MEDICAL DEVICE

(71) Applicant: NATIONAL TAIWAN UNIVERSITY, TAIPEI (TW)

(72) Inventors: Chih-Yu CHAO, TAIPEI (TW); Wei-Ting CHEN, TAIPEI (TW)

(21) Appl. No.: 14/306,235

(22) Filed: Jun. 17, 2014

(30) Foreign Application Priority Data
Dec. 11, 2013 (TW) 102145633

(51) Int. Cl. A61B 18/04

(52) U.S. Cl. CPC A61B 18/04; A61B 2018/00011

(57) ABSTRACT

An ultrasonic medical device includes a first ultrasonic transmitter, emitting a first ultrasonic with frequency f1, and a second ultrasonic transmitter, emitting a second ultrasonic with frequency f2. The pathways of these ultrasonics overlap at least a portion of each other in one medical treatment area, forming a first composite ultrasonic with frequency f3 in the medical treatment area. Both f1 and f2 are larger than f3. The composite ultrasonic can be used in medical treatment.

### RELATED APPLICATIONS

This application claims priority to Taiwanese Application Serial Number 102145633, filed Dec. 11, 2013, which is herein incorporated by reference.

### BACKGROUND

1. Field of Invention

The present invention relates to a medical device. More particularly, the present invention relates to an ultrasonic medical device.

2. Description of Related Art

Cancer, medically known as malignant neoplasm, is a broad group of diseases involving unregulated cells growth, including the normal cells transform to the cancer cells, which divide and grow uncontrollably, and forming malignant tumors. Further, the cancer may invade nearby parts of the body. The cancer may also spread to more distant parts of the body through the lymphatic system or bloodstream. Doctors can diagnose cancer by the tissue sample, or the amount of the biomarker from the patient. Once the diagnosis is confirmed, cancer is treated with chemotherapy, radiation therapy and surgery. With the improvement of science technology, many kinds of medicines focus on particular cancer to enhance the treating effects. Depending on their type, location in body, and development level, most of the cancer can be treated, even cured now.

In the field of malignant tumor treatment, traditional surgery, chemotherapy, and radiation therapy are therapies widely used now, however, each of them has its shortcomings and side effects. Therefore, a much more harmless and safer treatment to the patient is what many researchers focusing on. Thermal treatment and non-invasive therapy are current researching topics. The main principle of the thermal treatment is using a heat source to heat the cancer tissue to destroy the tumor cells. Usually, heating over about 50 to 54° C. makes protein denaturation. However, not all proteins in the tumor cells are denaturated and destroyed during the thermal treatment, so the tumor cells still have probability to relapse. Therefore, a long-duration-time heating process is needed to completely destroy the tumor cells. Once the thermal treatment takes a long heating time, the thermal energy may accumulate in body and harm organs.

The thermal treatment includes radio frequency tumor ablation (RFTA), microwave ablation therapy, and high-intensity focused ultrasound (HIFU) with different energy sources. RFTA and microwave ablation therapy still need to set a needle into a body, then heat the area around the needle. HIFU is a non-invasive treatment, which can treat tumor by concentrating the ultrasonic energy to raise the temperature at the focusing spot to destroy the tissue at the surrounding area. Because the HIFU needs to concentrate the ultrasonic from the body surface into the body, which makes HIFU has some operation limits. For example, HIFU needs to avoid bones for reflecting the ultrasonics. Nowadays, the ultrasonic intensity, which HIFU uses to burn the tumor is about 1 W/cm², the ultrasonic energy can be concentrated to 1,000 to 10,000 times of the original intensity. Therefore, the ultrasonic intensity at the focusing area can reach 1,000 W/cm² to 10,000 W/cm², and the contraction pressure of the ultrasonic wave may be about 30 MPa. The heating and burning may also harm the cells and tissue around the focus. Also, due to the device size and precision control limitation, HIFU cannot perform a precise treatment at the tumor boundary. Therefore, HIFU still has risk to patients, and the application is still limited to the tumor with large volume and low dangerousness such as hysteromyoma.

### SUMMARY

The present disclosure provides an ultrasonic medical device, which has no radiation damage, has no side effects what chemotherapy did, may define the boundary of tumor precisely, and may decrease the size of the device, cost and dangerousness. The medical device may not only apply in tumor treatment, but also can apply in other ways such as dissolving fat and medical cosmetology.

One aspect of the present disclosure is an ultrasonic medical device including a first ultrasonic transmitter, emitting a first ultrasonic with frequency f1; and a second ultrasonic transmitter, emitting a second ultrasonic with frequency f2; wherein the first and the second ultrasonic are both convergent ultrasonics, the pathways of these ultrasonics overlap with at least a portion of each other in a medical treatment area, forming a first composite ultrasonic with frequency f3 in the medical treatment area, both f1 and f2 are larger than f3, and the first composite ultrasonic has an average intensity larger than 10 W/cm².

In various embodiments of the present disclosure, the medical device further includes a third ultrasonic transmitter, emitting a third ultrasonic, wherein the third ultrasonic is a convergent ultrasonic, the pathway of the third ultrasonic overlaps at least a portion of the medical treatment area, forming a fourth composite ultrasonic together with the first and second ultrasonics in the medical treatment area, and the fourth composite ultrasonic has an average intensity more than 10 W/cm².

In various embodiments of the present disclosure, the above mentioned convergent ultrasonics are focused ultrasonics.

In various embodiments of the present disclosure, the medical device further includes a cooling device.

In various embodiments of the present disclosure, the above mentioned cooling device is selected from the group consisting of an ultrasonic waveform elimination device, a low temperature circulating cooling device, a thermoelectric cooling device, a local low temperature cooling kit and combinations thereof.

In various embodiments of the present disclosure, the above mentioned ultrasonic waveform elimination device includes at least one phase-oppositing ultrasonic transmitter, emitting at least one phase-oppositing ultrasonic to the medical treatment area and its peripheral.

In various embodiments of the present disclosure, the above mentioned ultrasonic waveform elimination device further includes at least one ultrasonic sensor, the ultrasonic sensor is used to sense a heat amplitude in the medical treatment area and its peripheral.

In various embodiments of the present disclosure, the above mentioned ultrasonic waveform elimination device further includes an ultrasonic analysis system, connecting with the ultrasonic sensor and the phase-oppositing ultrasonic transmitter.

In various embodiments of the present disclosure, the above mentioned emitting surface of the phase-oppositing ultrasonic transmitter has a circle-shape, ring-shape, polygon-shape and combinations thereof.

In various embodiments of the present disclosure, the above mentioned shape of the emitting surface of the phase-oppositing ultrasonic transmitter is a plurality of concentric rings, and the concentric rings emit different phase-oppositing ultrasonics to the medical treatment area and its peripheral from the small ring to the large ring simultaneously or sequentially.

In various embodiments of the present disclosure, the above mentioned low temperature circulating cooling device includes a circulation system, comprising a coolant flowing in the circulation system; a power device, mounted in the circulation system; and a heat sink, mounted in the circulation system.

In various embodiments of the present disclosure, the above mentioned thermoelectric cooling device includes: a thermoelectric cooling object; and a temperature adjusting system, connecting with the thermoelectric cooling object.

In various embodiments of the present disclosure, the above mentioned local low temperature cooling kit includes: a container, having a capacity space; and an endothermic substance, placed in the capacity space of the container.

In various embodiments of the present disclosure, the above mentioned container is setting on or under an operating table for decreasing the temperature of the affected area, medical treatment area and its peripheral.

In various embodiments of the present disclosure, the medical device further includes an automatic temperature control system including: an automatic control system, connecting with the cooling device; and a temperature sensor system, connecting with the automatic control system; wherein the automatic temperature control system controls the temperature of the cooling device.

In various embodiments of the present disclosure, the above mentioned automatic temperature control system combines with an operation control system to control the temperature in the medical treatment area during the operation, and can operate an automatic-controlling operation by the operation control system.

In various embodiments of the present disclosure, the above mentioned ultrasonic transmitter further includes a cooling attachment, the cooling attachment sets around the ultrasonic transmitter to form a low temperature ultrasonic transmitter.

In various embodiments of the present disclosure, a temperature in the medical treatment area is in a range from about 0° C. to about 54° C.

In various embodiments of the present disclosure, a temperature in the medical treatment area is in a range from about 0° C. to about 50° C.

In various embodiments of the present disclosure, a temperature in the medical treatment area is in a range from about 0° C. to about 45° C.

In various embodiments of the present disclosure, a temperature in the medical treatment area is in a range from about 0° C. to about 37° C.

In various embodiments of the present disclosure, the above mentioned average intensity of the first composite ultrasonic is more than 15 W/cm².

In various embodiments of the present disclosure, the above mentioned average intensity of the first composite ultrasonic is more than 20 W/cm².

In various embodiments of the present disclosure, the above mentioned average intensity of the first composite ultrasonic is more than 25 W/cm².

In various embodiments of the present disclosure, the above mentioned medical treatment area is set in a tumor or a fat tissue.

In various embodiments of the present disclosure, the above mentioned first composite ultrasonic resonates with the tissue in the medical treatment area, the average resonance intensity is larger than 10 W/cm².

In various embodiments of the present disclosure, the above mentioned first composite ultrasonic resonates with the tissue in the medical treatment area, the average resonance intensity is larger than 15 W/cm².

In various embodiments of the present disclosure, the above mentioned average resonance intensity is larger than 20 W/cm².

In various embodiments of the present disclosure, the above mentioned average resonance intensity is larger than 25 W/cm².

In various embodiments of the present disclosure, the above mentioned frequencies of the ultrasonics are less than 10 times of the frequency of the first composite ultrasonic.

In various embodiments of the present disclosure, the above mentioned first composite ultrasonic is a beat and its frequency f3=|f1−f2|.

In various embodiments of the present disclosure, the above mentioned first and second ultrasonics are both pulsed ultrasonics.

In various embodiments of the present disclosure, the pulse intensity, pulse duration time, and pulse frequency of the pulsed ultrasonics can be adjusted.

In various embodiments of the present disclosure, the above mentioned frequency of the first composite ultrasonic f3 is less than 200 kHz.

In various embodiments of the present disclosure, the above mentioned frequency of the first composite ultrasonic f3 is in a range from about 20 kHz to about 80 kHz.

In various embodiments of the present disclosure, the above mentioned frequency of the first composite ultrasonic f3 is in a range from about 20 kHz to about 60 kHz.

In various embodiments of the present disclosure, the above mentioned frequency of the first composite ultrasonic f3 is in a range from about 50 kHz to about 80 kHz.

In various embodiments of the present disclosure, the above mentioned frequency of the first composite ultrasonic f3 is in a range from about 150 kHz to about 200 kHz.

In various embodiments of the present disclosure, the frequency, intensity, and the convergency of the ultrasonics emitted by the ultrasonic transmitter can be adjusted.

In various embodiments of the present disclosure, the above mentioned frequency of the ultrasonic emitted by the ultrasonic transmitter is in a range from about 80 kHz to about 20MHz.

In various embodiments of the present disclosure, the above mentioned intensity of the ultrasonic emitted by the ultrasonic transmitter is in a range from about 1 mW/cm² to about 10 W/cm².

In various embodiments of the present disclosure, the cross sectional area of the first ultrasonic is larger, equal to, or smaller than the cross sectional area of the second ultrasonic.

In various embodiments of the present disclosure, the above mentioned focal length of the ultrasonic emitted by the ultrasonic transmitter can be adjusted.

In various embodiments of the present disclosure, the focus area of the ultrasonic emitted by the ultrasonic transmitter can be adjusted.

In various embodiments of the present disclosure, the spatial relationships between the ultrasonic transmitters can be defined by a perpendicular relative angle Φ and a horizontal relative angle φ.

In various embodiments of the present disclosure, the medical device further includes a magnetic resonance imaging or an ultrasonic tomography to define the affected area.

In various embodiments of the present disclosure, the medical device further includes an integrated operation control system, the system can control and adjust the locations of the ultrasonic transmitters, the emitting directions of the ultrasonics, and the frequencies and intensities of the ultrasonics emitted by the ultrasonic transmitters.

In various embodiments of the present disclosure, the medical device further includes a composite probe, the composite probe having an emitting surface, wherein the ultrasonic transmitters mount on the emitting surface of the composite probe, and the distance between the ultrasonic transmitters and the emitting angles of the ultrasonic transmitters on the composite probe can be adjusted.

In various embodiments of the present disclosure, the above mentioned emitting surface of the composite probe is a curved surface.

In various embodiments of the present disclosure, the medical device further includes a water bag disposed between the composite probe and the medical treatment area.

Another aspect of the present disclosure is an ultrasonic temperature controlling method for eliminating a heat generated from a heat generation area, including; sensing the first heat amplitude generating from the heat generation area and its peripheral, and analyzing the waveform of the first heat amplitude; and emitting a first phase-oppositing ultrasonic having an out-of-phase waveform in relation with the first heat amplitude to the heat generation area and its peripheral.

In various embodiments of the present disclosure, the method further includes: sensing the second heat amplitude generating from the heat generation area and its peripheral, and analyzing the waveform of the second heat amplitude, wherein the second heat amplitude is the residue of the first heat amplitude: and emitting a second phase-oppositing ultrasonic having an out-of-phase waveform in relation with the second heat amplitude to the heat generation area and its peripheral.

In various embodiments of the present disclosure, the operation emitting a first phase-oppositing ultrasonic having an out-of-phase waveform in relation with the first heat amplitude to the heat generation area and its peripheral includes emitting a plurality of phase-oppositing ultrasonics with different frequencies to the heat generation area and its peripheral.

In various embodiments of the present disclosure, the operation emitting a first phase-oppositing ultrasonic having an out-of-phase waveform in relation with the first heat amplitude to the heat generation area and its peripheral includes emitting a plurality of phase-oppositing ultrasonics with different amplitudes to the heat generation area and its peripheral.

in various embodiments of the present disclosure, the operation emitting a first phase-oppositing ultrasonic having an out-of-phase waveform in relation with the first heat amplitude to the heat generation area and its peripheral includes emitting a plurality of phase-oppositing ultrasonics to the different parts of the heat generation area and its peripheral.

It is to be understood that both the foregoing general description and the following detailed description are by examples, and are intended to provide further explanation of the invention as claimed.

### BRIEF DESCRIPTION OF THE DRAWINGS

Aspects of the present disclosure are best understood from the following detailed description when read with the accompanying figures. It is noted that, in accordance with the standard practice in the industry, various features are not drawn to scale. In fact, the dimensions of the various features may be arbitrarily increased or reduced for clarity of discussion.

FIG. 1A is a schematic diagram of wave interference according to various embodiments of the present disclosure;

FIG. 1B is a schematic diagram of beat according to various embodiments of the present disclosure;

FIG. 1C is a schematic diagram of wave resonance according to various embodiments of the present disclosure;

FIG. 2 is a schematic diagram of a relationship between ultrasonic intensity and frequency according to various embodiments of the present disclosure;

FIG. 3 is a schematic diagram of an ultrasonic medical device according to various embodiments of the present disclosure;

FIG. 4A is a schematic diagram of an ultrasonic medical device according to various embodiments of the present disclosure;

FIG. 4B is a schematic diagram of an ultrasonic medical device according to various embodiments of the present disclosure;

FIG. 5 is a schematic diagram of an operation coordinate of an ultrasonic medical device according to various embodiments of the present disclosure;

FIG. 6A is a schematic diagram of an operation of ultrasonic medical device according to various embodiments of the present disclosure;

FIG. 6B is a schematic diagram of an operation of ultrasonic medical device according to various embodiments of the present disclosure;

FIG. 6C is a schematic diagram of an operation of ultrasonic medical device according to various embodiments of the present disclosure;

FIG. 6D is a schematic diagram of an operation of ultrasonic medical device according to various embodiments of the present disclosure;

FIG. 7 is a schematic diagram of an ultrasonic medical device according to various embodiments of the present disclosure;

FIG. 8A is a schematic diagram of an ultrasonic medical device according to various embodiments of the present disclosure;

FIG. 8B is a schematic diagram of an ultrasonic medical device according to various embodiments of the present disclosure;

FIG. 9 is a schematic diagram of a phase-oppositing ultrasonic transmitter of an ultrasonic medical device according to various embodiments of the present disclosure;

FIG. 10 is a schematic diagram of a low temperature circulating cooling device of an ultrasonic medical device according to various embodiments of the present disclosure;

FIG. 11A is a schematic diagram of a low temperature ultrasonic transmitter of an ultrasonic medical device according to various embodiments of the present disclosure;

FIG. 11B is a schematic diagram of an ultrasonic medical device according to various embodiments of the present disclosure;

FIG. 12A is a schematic diagram of an ultrasonic medical device according to various embodiments of the present disclosure;

FIG. 12B is a schematic diagram of an ultrasonic medical device according to various embodiments of the present disclosure;

FIG. 13A is a schematic diagram of a composite probe of an ultrasonic medical device according to various embodiments of the present disclosure;

FIG. 13B is a schematic diagram of a composite probe of an ultrasonic medical device according to various embodiments of the present disclosure;

FIG. 13C is a schematic diagram of a composite probe of an ultrasonic medical device according to various embodiments of the present disclosure; and

FIG. 14 is a schematic diagram of a composite probe of an ultrasonic medical device according to various embodiments of the present disclosure.

### DETAILED DESCRIPTION

Reference will now be made in detail to the present embodiments of the invention, examples of which are illustrated in the accompanying drawings. Wherever possible, the same reference numbers are used in the drawings and the description to refer to the same or like parts.

The following disclosure provides many different embodiments, or examples, for implementing different features of the provided subject matter. Specific examples of components and arrangements are described below to simplify the present disclosure. These are, of course, merely examples and are not intended to be limiting. For example, the formation of a first feature over or on a second feature in the description that follows may include embodiments in which the first and second features are formed in direct contact, and may also include embodiments in which additional features may be formed between the first and second features, such that the first and second features may not be in direct contact. In addition, the present disclosure may repeat reference numerals and/or letters in the various examples. This repetition is for the purpose of simplicity and clarity and does not in itself dictate a relationship between the various embodiments and/or configurations discussed.

As used herein, the terms “comprising,” “including,” “having,” “containing,” “involving,” and the like are to be understood to be open-ended, i.e., to mean including but not limited to.

The singular forms “an” and “the” used herein include plural referents unless the context clearly dictates otherwise. Therefore, reference to, for example, a dielectric layer includes embodiments having two or more such dielectric layers, unless the context clearly indicates otherwise. Reference throughout this specification to “one embodiment” or “an embodiment” means that a particular feature, structure, or characteristic described in connection with the embodiment is included in at least one embodiment of the present disclosure. Therefore, the appearances of the phrases “in one embodiment” or “in an embodiment” in various places throughout this specification are not necessarily all referring to the same embodiment. Further, the particular features, structures, or characteristics may be combined in any suitable manner in one or more embodiments. It should be appreciated that the following figures are not drawn to scale; rather, these figures are intended for illustration.

### Disclosure Principle

A widely investigation has been made for designing an invasive or non-invasive medical device base on ultrasonic principles. The present disclosure is based on ultrasonic properties and principles, further using many special properties of composite ultrasonics to investigate an ultrasonic medical device, which is a noninvasive device and can decrease dangerousness of operations.

The principle of the ultrasonic medical device disclosed herein is using the special property of an ultrasonic that the ultrasonics can form mechanical resonance with body tissue at specific frequency. When the ultrasound has enough intensity (Iᵣₘₛ²), the resonating body tissue may be destroyed. But if the ultrasonic with specific frequency and intensity is directly emitted from the outside of the body to a target tissue inside the body, the tissue along the ultrasonic pathway would be destroyed. The present disclosure uses the ultrasonic superposition property to design an ultrasonic medical device, which can operate in a harmless way by emitting two extracorporeal ultrasonics, which may not destroy body tissue, and let the two ultrasonics intersect at an unwanted body tissue to form a composite ultrasonic that can resonate with the tissue to destroy it. According to various embodiments of the present disclosure, the unwanted body tissue is a tumor. In some embodiments, the unwanted body tissue is fat. In some embodiments, the composite ultrasonic can be tuned to appropriate intensity and frequency, to apply the ultrasonic medical device to massage and stimulate the blood circulation of body without destroying the tissue. Following are detailed description of the disclosure principle, for further understanding for the concept of present disclosure.

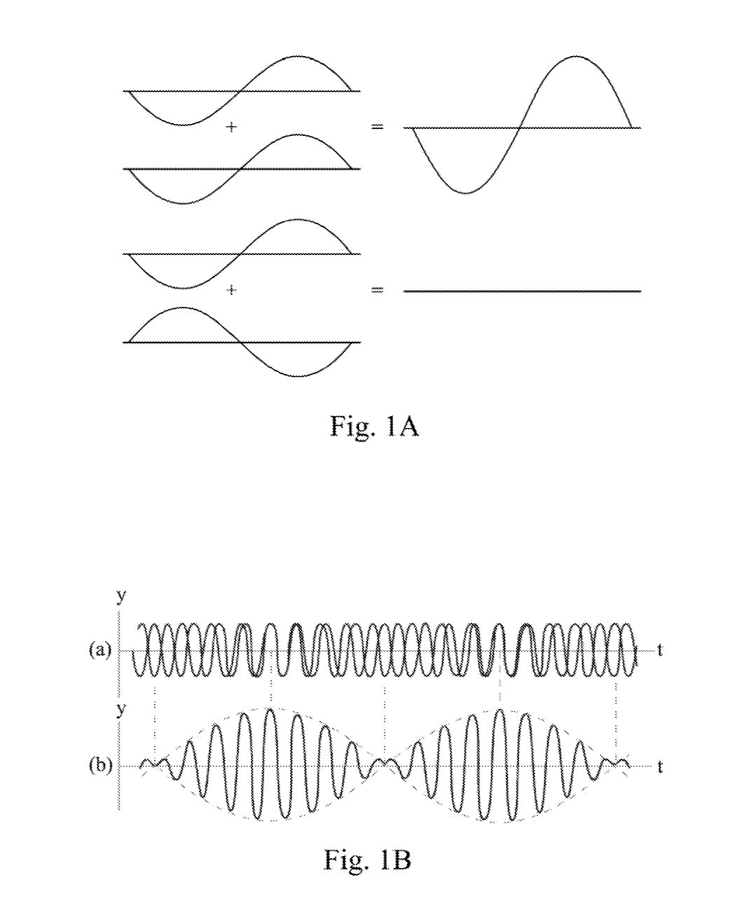

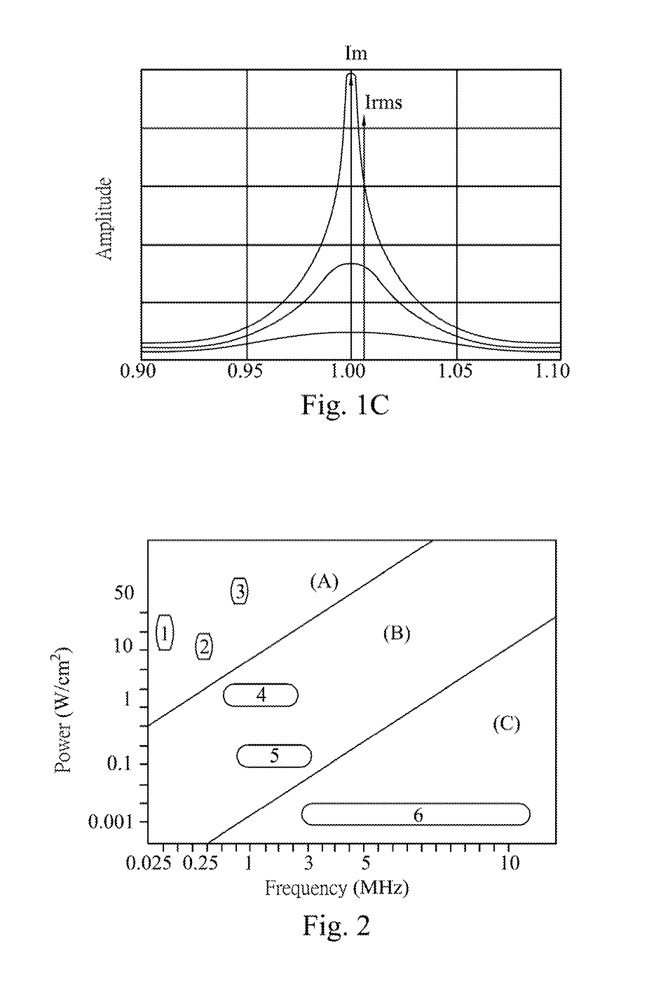

Referring to FIG. 1A, FIG. 1A depicts two ultrasonics forming a composite ultrasonic with constructive and destructive interference when the two ultrasonics are overlapped. This is a basic property of ultrasonics, when two ultrasonics overlap, interference may happen and form a composite ultrasonic. When the two ultrasonics have the same frequency and phase, which are in phase, the composite ultrasonic may have maximum amplitude, generating the constructive interference, as the upper line in the figure. When the two ultrasonics have the same frequency and amplitude but opposite phase, which are out of phase, an amplitude of the composite ultrasonic will be cancelled, generating the destructive interference, as the lower line in the figure.

Beat forms when the two ultrasonics have close frequency interfering with each other. Referring to FIG. 1B, FIG. 1B is a schematic diagram of two ultrasonics with the same amplitude and different frequencies forming a beat. In FIG. 18, an upper y-t diagram overlaps two ultrasonics with the same amplitude and close frequency, in which the y-t diagram represents the relationship of the medium particles oscillating with time at one place. A lower y-t diagram depicts a lower frequency composite ultrasonic formed by the two ultrasonics in the upper y-t diagram. FIG. 18 shows how the two ultrasonics are superposed to form the composite ultrasonic, and the relationship between amplitude and time of the composite ultrasonic. The frequency of the beat ultrasonic is the frequency difference between the two ultrasonics. And the maximum amplitude of the beat ultrasonic is twice of the amplitude of the former ultrasonics. The concept of superposing at least two ultrasonics to form a composite ultrasonic with lower frequency is useful in the present disclosure. A non-invasive and less-dangerous ultrasonic medical device may be made by utilizing the above mentioned property. By using high frequency ultrasonics, which will not destroy tissue, to form a low frequency ultrasonic in the place where needs a treatment. Although in FIG. 1B shows the beat, but the present disclosure is not limited to using ultrasonic having beat frequency, any lower frequency ultrasonics superposed by at least two higher frequency ultrasonics are included in the present disclosure.

When ultrasonic interacts with body tissue, part of the energy may transform to the body tissue and be absorbed. The absorbed energy may have different physical effect depending on a sum of the absorbed energy, an absorption rate of the energy, and a property of the body tissue. The main physical effect includes generating heat, mechanical effect, and cavitation effect. Heat generation is the main principle used by HIFU, which focus the ultrasonic to generate high energy to burn the body tissue. When the temperature arises to about 50 to about 54° C., proteins may start to denature and solidify, and when the power of the ultrasonics is over 1000 W/cm², the body tissue may be destroyed.

Cavitation effect is bubbles forming in the body tissue, which includes stable bubble and transient bubble. Usually the stable bubble forms when the ultrasonic power is smaller, usually lower than 10 W/cm². The stable bubble mainly oscillates around the equilibrium point, destroying only local points. The transient bubble forms in a higher ultrasonic power condition, usually higher than 10 W/cm². The transient bubble may become larger and unstable in a short period of time and then explode and collapse. The large energy released by the explosion also disrupts the tissue around. A common application of cavitation effect is adding artificial bubbles to enhance amplitude when the bubbles oscillating, using the bubbles to break through a blood vessel barrier for drug delivering.

Mechanical effect is the main principle of the present disclosure, which is a substance tissue destroyed by changing particle displacements in the substance. In various embodiments of the present disclosure, further using the low frequency composite ultrasonic to resonate with the body tissue to enhance the amplitude made by the mechanical effect. In some embodiments, the mechanical amplitude may destroy distinct area such as tumor tissue.

The above mentioned three effects may all happen during the energy transfer process. The present disclosure mainly uses the mechanical effect, and the heat generation in the process is much smaller than HIFU, which is base on generating heat as the main treatment. And the heat generation is not necessary in the present disclosure. Therefore, in various embodiments of the present disclosure, a method of cooling by ultrasonic, and a cooling device, which may decrease the temperature in an area, where the composite ultrasonic forms, and its peripheral, are provided. The ultrasonic medical device may enhance the accuracy of controlling, and eliminate the heat damage of the tissue, which the medical treatment operates, and its peripheral.

An ultrasonic intensity may also affect the efficiency of the medical device. Although a low frequency ultrasonic may resonate with the body tissue in a to specific frequency to enhance an amplitude of a mechanical wave, as illustrated in FIG. 1C. FIG. 1C is a schematic diagram of a resonance intensity. As shown in FIG. 1C, the maximum amplitude Iₘ may exist at a resonance frequency, the root mean square amplitude Iᵣₘₛ is also shown on the figure. When damping in the system is smaller, resonance amplitude is larger. The magnitude of the amplitude of three curves in the figure from low to high is caused by the damping from high to low. Energy may transfer in an easiest way when resonance occurs. However, the composite ultrasonic still needs enough intensity to transfer the energy to the body tissue in order to destroy the tumor, as described in some embodiments. The cavitation effect may occur and join the mechanical effect to destroy the medical treatment area when the composite ultrasonic resonates in the area. When defining the ultrasonic intensity used in the present disclosure, following phenomena needs to be considered. The different part of the tissue in the medical treatment area may have bubbles in different sizes, so the tissue in different space (r) may reflect different oscillating amplitude I(r). Also when in different time (t), the tissue may have its own oscillating amplitude I(t), as the following formula:

$$\begin{matrix}Irms = \sqrt{\frac{1}{λ} {\int }_{0}^{λ} {[ I ( r ) ]}^{2} d r} & ( 1 ) \\ Irms = \sqrt{\frac{1}{T} {\int }_{0}^{T} {[ I ( t ) ]}^{2} d t} & ( 2 ) \\ Irms = \sqrt{\frac{1}{T} {\int }_{0}^{T} {[ \frac{1}{λ} {\int }_{0}^{λ} {I ( r , t )}^{2} d r ]}^{2} d t} & ( 3 )\end{matrix}$$

Formula 1 shows a spatial average of the root mean square amplitude, formula 2 shows a time average of the root mean square amplitude, and formula 3 shows a time and spatial average of the root mean square amplitude. To square the time and spatial average of the root mean square amplitude can have an average intensity, as follows:

I=Iᵣₘₛ²=Iₘₐₓ²/2 (4)

Formula 4 shows the ultrasonic intensity definition for tissue in the present disclosure.

And the ultrasonic emitted by the ultrasonic transmitter can use the definition of spatial average (SA), because the emitted ultrasonic would concentrate in the central area as a well known property. In the other aspect, to describe the temporal average intensity of the ultrasonic, the definition of to temporal average (TA) is also commonly used. Besides, if the intensity definition of the ultrasonic is temporal peak (TP) or pulse average (PA), the numbers can be transformed to TA by a duty factor (DF), as the following formula:

DF=pulse (peak) duration time×pulse (peak) occurrence frequency (5)

TA=DF_{P}×PA (6)

TA=DF_{T}×TP (7)

The ultrasonic makes no damage to human body when the frequency is high and the intensity is low, like diagnosis ultrasound. But when the ultrasonic is in low frequency, once the intensity is high, medium may oscillate fiercely. If the ultrasonic frequency is the specific frequency, which may resonate with the medium,the medium structure may be destroyed. Therefore, the intensity and frequency of the ultrasonic are both important conditions to destroy the medium structure. Both “having right frequency but without enough intensity” and “having enough intensity but with wrong frequency” may not resonate with the medium to destroy the medium structure, such as tissue. For example, human to cells may resonate with ultrasonics having frequency in a range from 20 to 60 kHz When the ultrasonic has enough intensity, which means the resonance amplitude is large enough, the cells and tissue may be destroyed and become emulsification. In recent years, a higher ultrasonic frequency range up to 55 kHz used on the boundary of tissue and device also has good cutting and condensing effect. The character of ultrasonic intensity can be described as following formula:

I=W/A=½×ρ×v×ζ²×ω² (8)

In the formula, I is the ultrasonic intensity; W is power; A is area; is medium density; v is ultrasonic velocity; is the ultrasonic amplitude in the medium, is angular frequency which equals to 2 f, in which f is ultrasonic frequency.

From formula 8, when the ultrasonic intensity is fixed, the higher the ultrasonic frequency, the smaller the amplitude in the medium; in other words, the lower the ultrasonic frequency, the larger the amplitude in the medium.

In various embodiments of the present disclosure, in order to make sure the composite ultrasonic may transfer enough energy for amplitude) to resonate with the cells, following formula is derived by using following rules. When beat forms, the amplitude of beat is twice of the amplitude of the two ultrasonics Based on formula 8, substitute ultrasonics with their intensities whose resultant beat intensity (with right frequency to resonate with cells) is large enough to destroy tissue, and using the conditions that beat intensity is only twice of the two ultrasonics and the beat frequency is the frequency difference of the two ultrasonics, the following formula 9 can be derived using these conditions. By adjusting the frequency of the two ultrasonics may derive the intensity of the two ultrasonics, and find that the calculated ultrasonic intensity is larger than the beat intensity. Most of the intensity difference is transferred to heat energy. By the above calculation, an energy transfer ratio η can be calculated by dividing beat intensity with a sum of the two ultrasonic intensities. And η is found to have an relationship with the frequency ratio x of two ultrasonics and beat as following:

η∝1/x² (9)

Formula 9 represents that the larger the frequency ratio of two ultrasonics and beat, the smaller the energy transfer ratio. In other words, the larger the frequency ratio is, the more heat generating when the beat reaches enough intensity (Iᵣₘₛ²) to destroy the tissue in the medical treatment area. The heat generated here is still much smaller than the heat that the HIFU produces in the focal area, but still needs to prevent the tissue temperature from rising to over 54° C. which causes the protein denature. In various embodiments of the present disclosure, two ultrasonics are pulsed ultrasonics, making the generated heat has enough time to dissipate to let the tissue not injured by heat. According to some embodiments of the present disclosure, the ultrasonic medical device further includes a cooling device, which can lower the temperature in the medical treatment area, and thus makes the heat generated during ultrasonic energy transfer not affecting the tissue in the medical to treatment area and its peripheral. Moreover, the ultrasonic medical device also can enlarge the frequency ratio between the two ultrasonics and the beat to enhance the ultrasonic frequency range in the present disclosure. The broaden range the ultrasonic frequency may use, the safety and the utility of the medical device may be enhanced. Therefore, in some embodiments of the present disclosure, an ultrasonic cooling method is provided.

Using the above mentioned principles, in various embodiments of the present disclosure, using two ultrasonics with frequency difference to form a medical treatment area at the intersection of the pathways of the two ultrasonics. The two ultrasonics superpose a lower frequency composite ultrasonic in the medical treatment area, the amplitude of the composite ultrasonic is the sum of the amplitudes of the two ultrasonics in the medical treatment area; the intensity of the composite ultrasonic is the square of the amplitude of the composite ultrasonic; and the frequency of the composite ultrasonic equals to the frequency difference of the two ultrasonics. Therefore adjusting the frequency and intensity of the ultrasonics may let the medium resonate and thus form intense oscillating by mechanical and cavitation effect and then destroyed. The cavitation is naturally formed in the body tissue, and is induced by ultrasonic with frequency lower than 80 kHz, not by swallowing or injecting artificial bubbles, which is used for ultrasonic imaging, drug delivery or cavitation effect. The composite ultrasonic may destroy cells and tissue to treat tumor, and dissolve fat at different frequency and intensity. Therefore a non-invasive treatment may be realized by using the ultrasonic medical device. The device only needs to adjust the ultrasonic pathway and a convergence of the ultrasonic at outside of the body, and makes the two ultrasonic pathways intersect in an affected area. These two ultrasonics can generate the composite ultrasonic with proper frequency and enough intensity to destroy the cells or tissue in the distinct affected area. Therefore the ultrasonic medical device may provide a safe, without radiation and heat damage, and can conduct precisely controlled operation, which can apply for tumor treatment to destroy tumor tissue. The following description will further explain the various embodiments and applications of the ultrasonic medical device in the present disclosure.

### DETAILED DESCRIPTION

Referring to FIG. 2, FIG. 2 is a schematic diagram of a relationship between ultrasonic intensity and frequency according to various embodiments of the present disclosure. The figure is based on US Food and Drug Administration (FDA) to show an effect of ultrasonics with different intensity and frequency. The figure is described in logarithmic scale. There are three big regions in the figure, when the ultrasonics have frequency and intensity in region A, the ultrasonics have mechanical treatment effect including lithotripsy and lipolisis. Section 1 shows the ultrasonic frequency and intensity range for Ultrasonic Assisted Liposuction (UAL); section 2 shows the ultrasonic frequency and intensity range for Ultrashape; and section 3 shows the ultrasonic frequency and intensity range for extracorporeal shock wave lithotripsy (ESWL). Region B is the thermal therapeutic region, including section 4 for HIFU and section 5 for physiotherapy. Region C is diagnostic region, including section 6 with ultrasonic frequency and intensity range for imaging. The ultrasonic frequency and intensity in region B and C are harmless to human body. The figure also shows that when the ultrasonic intensity is the same, the lower the ultrasonic frequency, the larger the amplitude of the medium, and the application of the ultrasonic is based on mechanical effect; when the ultrasonic frequency is the same, the ultrasonic intensity is larger, which also can have the same effect. In some embodiments of the present disclosure, the principle is used. For example, the two ultrasonics have frequency about 500 kHz, frequency difference about 50 kHz, and intensity are both 0.3 W/cm², Referring to FIG. 2, the energy of two ultrasonics are both in the region B, which means no harm for people. In this time, only adjusting the ultrasonic pathways and the convergence of the ultrasonics from the extracorporeal can let them intersect at the affected area. A composite ultrasonic may be formed when the two ultrasonics intersect, the composite ultrasonic has frequency about 50 kHz, and the intensity is larger than 10 W/cm². Referring to FIG. 2, the frequency and intensity of the generated composite ultrasonic lies in region A, meaning that the composite ultrasonic may make mechanical harming to the human cells and tissue. The frequency difference of the two ultrasonics may be adjusted continuously to superpose a low frequency composite ultrasonic with a wanted intensity. In operation, the ultrasonic intensity, the convergence of the ultrasonics, or both may be adjusted. For example, a composite ultrasonic with frequency about 25 kHz and intensity still larger than 10 W/cm² may be superposed. The composite ultrasonic with much lower frequency still has good effect for trimming, condensing, and emulsifying tissue. In various embodiments, the properties are used to manufacture a medical device, which may destroy tissue in specific portion. The cells on only one ultrasonic pathway won't get any harm, only tissue on the intersection area of the two ultrasonics may be destroyed by the composite ultrasonic. And leave no sequelae to body. The medical device may apply for non-invasive tumor treatment and have no side effect.

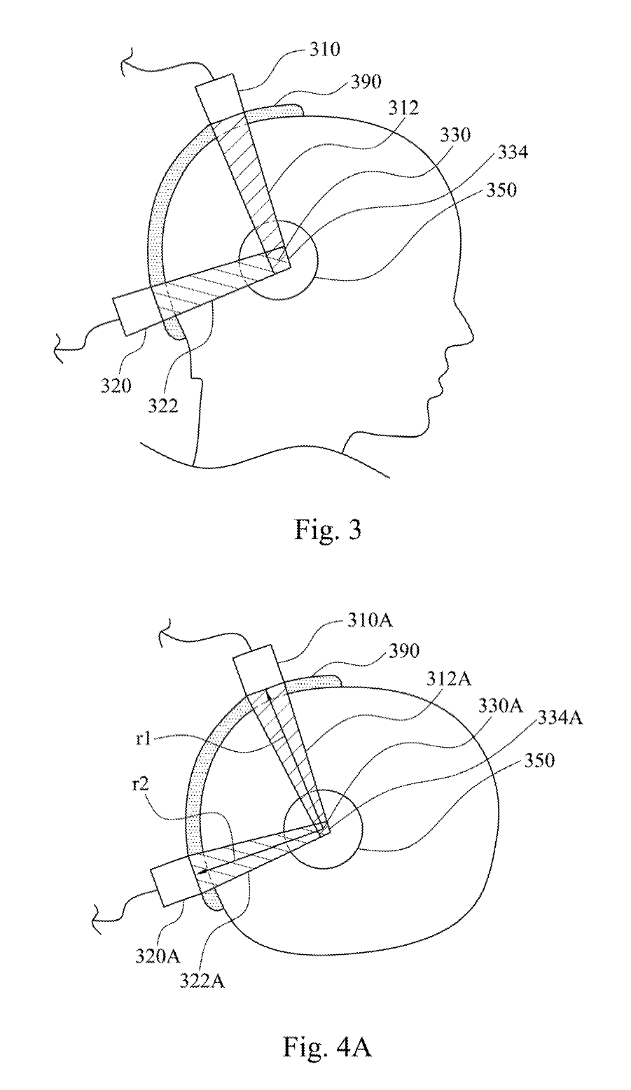

Referring to FIG. 3, FIG. 3 is a schematic diagram of an ultrasonic medical device according to various embodiments of the present disclosure. The ultrasonic medical device includes a first ultrasonic transmitter 310 emitting a first ultrasonic 312 with frequency f1, intensity E1 and area A1. A second ultrasonic transmitter 320 emits a second ultrasonic 322 with frequency f2, intensity E2 and area A2. In some embodiments of the present disclosure, the ultrasonic transmitters 310, 320 separately emit two ultrasonics 312, 322, which have no harm to human body. The pathways of these ultrasonics 312, 322 overlap at least a portion of each other to form one medical treatment area 334, and form a first composite ultrasonic 330 with frequency f3 in the medical treatment area. The composite ultrasonic 330 may destroy tissue in the medical treatment area. For example, the frequency f1, f2 of the two ultrasonics are about 550 kHz, and have frequency difference about 60 kHz, and their intensity are both about 0.5 W/cm². When operating, both the position of the ultrasonic transmitters 310, 320 and the convergence of the ultrasonics 312, 322 may be adjusted. The composite ultrasonic may be superposed in the medical treatment area, in which the frequency of the composite ultrasonic is 60 kHz, and the intensity is over 10 W/cm². And the composite ultrasonic may mechanically harm the human tissue and cells. In other words, the two ultrasonics 312, 322 may form a first composite ultrasonic 330 with frequency f3 and enough intensity, both f1 and f2 are larger than f3. The frequency f3 and the intensity of the first composite ultrasonic 330 may destroy the tissue and cells in the medical treatment area 334, where has the first composite ultrasonic 330. The medical treatment area 334 is settled in the affected area 350. In some embodiments of the present disclosure, the affected area is a tumor. In various embodiments of the present disclosure, the first ultrasonic 312 and the second ultrasonic 322 are both convergence ultrasonics. In various embodiments of the present disclosure, the ultrasonics 312, 322 emitted by the ultrasonic transmitters 310, 312 are both pulsed ultrasonics. The pulsed ultrasonics may largely decrease the energy absorption from the two ultrasonics to the tissue in the medical treatment area. Thus, the pulsed ultrasonics can make the heat generated in the medical treatment area has enough time to dissipate. However, the pulsed ultrasonics may not affect the tissue to absorb resonance energy caused by the first composite ultrasonic 330 resonating with the tissue. Once the pulsed ultrasonic energy passing by the medical treatment area 334, the energy of the first composite ultrasonic 330 may almost be completely absorbed by resonance. Therefore the method may avoid the tissue around the medical treatment area 334 get injured by heat. In various embodiments of the present disclosure, the intensity of the first composite ultrasonic 330 is about 10 W/cm², but the intensity of each ultrasonic 312, 322 around the intersection of the pathways may reach about 200 W/cm². In comparison with the tissue burning (>54° C.) which needs intensity about 1000 W/cm², the intensity of the composite ultrasonic 330 is less prone to generate heat damage to the medical treatment area and its peripheral. Changing each ultrasonics 312, 322 to pulsed ultrasonics, adjusting the pulse intensity, pulse duration time, and pulse frequency, may decrease the two ultrasonic energy absorbed by the tissue in to the medical treatment area 334 for 3 to 5 times or more. Therefore the tissue is less prone to get heat injury. In various embodiments of the present disclosure, under the human body temperature 37° C., the frequency of the ultrasonics may be less than 10 times of the composite ultrasonic.

The intensity of each ultrasonic may be adjusted, therefore the area of is the first ultrasonic transmitter A1 may be larger, equal to, or smaller than the area of the second ultrasonic transmitter A2. Further, the tissue and cells in the medical treatment area 334 may have resonance absorbing with the first composite ultrasonic, which has enough intensity and frequency f3. The energy of the first composite ultrasonic 330 may mostly transfer to the tissue in the medical treatment area by resonance, to form mechanical effect, cavitation effect or generating heat. In the mean while, part of the energy of the previous two ultrasonics may also transfer to the tissue in the area, mainly forming heat effect. The remaining energy of the two ultrasonics may scatter behind the medical treatment area, which thus makes no harm to the cells and tissue behind the area. In various embodiments of the present disclosure, an ultrasonic conductor 390 may dispose between the ultrasonic transmitters 310, 320 and a treatment body, which includes the affected area 350. To let the ultrasonics enter the treatment body through the ultrasonic conductor 390 to the affected area 350. The ultrasonic conductor 390 may include oil, water, emulsion, gel, or combinations thereof. Because the ultrasonic transmitter is a well-known device, therefore the ultrasonic generation principle and the electric circuit may not be further described in the present disclosure. The other parts of the ultrasonic transmitter also are neglected in the figures. What is useful is how to choose and operate the ultrasonics generated by the ultrasonic transmitters to form the composite ultrasonic and have a treatment effect. In various embodiments of the present disclosure, the intensity of the composite ultrasonic is larger than 10 W/cm², for example, 15, 20, and 25 W/cm². The critical intensity 10 W/cm² is the intensity that a transit bubble may form in the tissue. In a microscopic system, an explosion of the transit bubble may destroy the tissue and motivate the resonance intensity in the tissue. In various embodiments of the present disclosure, when the resonance intensity of the tissue in the medical treatment area is larger than 10 W/cm², for example, 15, 20, and 25 W/cm², the cells and tissue are absorbing enough energy to resonate by cavitation effect and mechanical effect to destroy the tissue in the medical treatment area.

Referring to FIG. 3, in various embodiments of the present disclosure, the first composite ultrasonic 330 formed in the intersection of the two ultrasonics 312, 322 is beat, and f3=|f1−f2|. In various embodiments of the present disclosure, the composite ultrasonic may not be beat, only needs two ultrasonics superposing to form a composite ultrasonic with frequency different from the two ultrasonics. And the two ultrasonics may not affect humans or animal tissue or cells. The composite ultrasonic may destroy the cells and tissue depending on the frequency and intensity.

Further, additional information is described in the following, even the frequency of the composite ultrasonic is disclosed as f3=|f1−f2|. Because human body, which includes muscles, organs, bones and tissue, may have damping when the ultrasonics pass through the human body. The ultrasonic frequency arriving the affected area f1′ may be smaller than the frequency f1 generated out of the body. But the decreased frequency may be a little small in comparison with the frequency f1 itself, therefore the effectiveness of the medical device may not be affected. However, because each of the frequency f1 and f2 may decay with different level, the frequency f1 may decay to f1′ and f2 may decay to f2′. The frequency of the composite ultrasonic f3 in fact is the difference of the two decayed ultrasonics f1′−f2′ But the value of f1′−f2′ may be close to the value of f1−f2, or still have a little difference. So the frequency of the ultrasonic f1, f2 should be properly adjusted to reach the purpose f3. In order to accord with the impression that the frequency will not change, and not to get confused, the frequency f1′ and f2′ may not be further mentioned in the present disclosure, only use f1 and f2 representing the frequency of the ultrasonics. Also when there is frequency deviation in operating, the frequency of the two ultrasonics till may be adjusted in anytime to fit the frequency of the composite ultrasonic to have treatment effect.

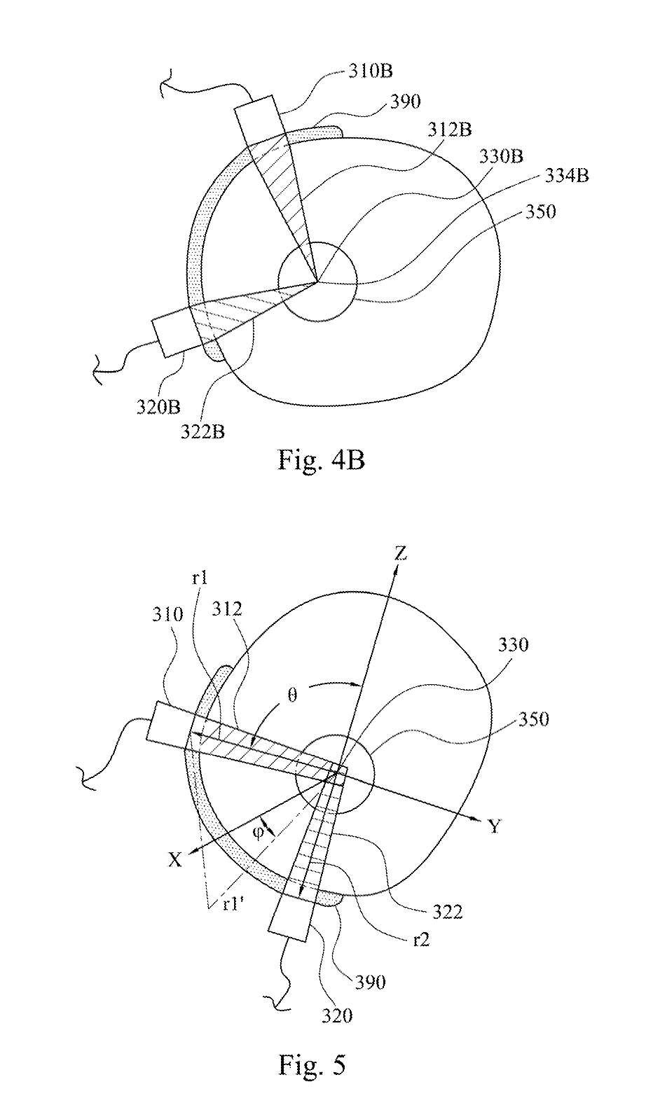

Referring to FIG. 4A, 4B, FIG. 4A, 4B are schematic diagrams of an ultrasonic medical device according to various embodiments of the present disclosure. In one embodiment depicted in FIG. 4A, using two ultrasonics 312A, 322A, which have no harm to human body. For example, the two frequencies of the ultrasonics f1, f2 are about 1 MHz, and the difference of the two frequencies is 55 kHz; the intensities are both about 0.75 W/cm². The location and emitting direction, focal length r1, r2 of the two ultrasonic transmitters 310A, 320A, the convergence of the two ultrasonics and the focus area of the two ultrasonics may be adjusted. Making at least part of the pathways of the ultrasonics 312A, 322A intersect with each other to form the medical treatment area 334A. In various embodiments of the present disclosure, the two ultrasonics are both pulsed ultrasonics, in which the pulse intensity, pulse duration time, and pulse frequency of the pulsed ultrasonic may be adjusted to decrease the two ultrasonic energy absorbed by the tissue in the medical treatment area 334A for 3 to 5 times or more. In various embodiments of the present disclosure, the energy of the two ultrasonics 312A, 322A absorbed by the tissue may further decrease for 5 to 10 times or more. The pulsed ultrasonic may increase the thermal dissipation rate of the tissue, making an overmuch heat be absorbed quickly and efficiently by the whole tissue. Therefore the tissue may not have much heat injury. A low frequency composite ultrasonic with frequency 55 kHz and intensity larger than 10 W/cm² may be formed in the medical treatment area. The composite ultrasonic with low frequency and high intensity may have mechanical injury to human tissue or cells. In other words, the two ultrasonics 312A, 322A may form a first composite ultrasonic 330 in the pathway intersection, and both f1 and f2 are larger than f3. The frequency f3 and the intensity of the first composite ultrasonic 330A may destroy the tissue and cells in the medical treatment area 334A. In various embodiments of the present disclosure, the frequency of the two ultrasonics may be smaller than 20 times of frequency of the composite ultrasonic under human body temperature 37° C.

Referring to FIG. 4B, FIG. 4B is schematic diagrams of an ultrasonic medical device according to various embodiments of the present disclosure. As depicted in the FIG. 4B, the ultrasonic medical device includes a first ultrasonic transmitter 310B emitting a first ultrasonic 312B with frequency f1 and intensity E11. A second ultrasonic transmitter 320B emits a second ultrasonic 322B with frequency f2 and intensity E2. In some embodiments of the present sir disclosure, the two ultrasonics 312B, 322B are focus beams. Also referring to FIG. 2, the intensity of the ultrasonic of the HIFU is about 1W/cm². HIFU can focus the ultrasonic to make the ultrasonic intensity at the focus point reach to 1,000 to 10,000 times of the original intensity to burn the tissue, which makes the tissue condensed or becomes necrosis. However, the ultrasonic medical device in the present disclosure may form the mechanical injury to destroy or emulsify the human tissue or cells only by using the low frequency composite ultrasonic or beat in the intensity about 10 to 30 W/cm² For example, using the two ultrasonics with frequency f1, f2 about 800 kHz, and the difference of the two ultrasonics is 55 KHz. As the focused ultrasonics may focus the ultrasonic intensity to at least 1,000 to 10,000 times, the intensities of the two ultrasonic 312B, 322B only need about 50 mW/cm² to about 0.5 W/cm²to form a low frequency composite ultrasonic with frequency about 55 kHz and intensity larger than 10 W/cm². The traditional HIFU, which only depends on heat generation, has the ultrasonic intensity about 1,000 W/cm² to 10,000 W/cm² at the focused area, and may burn the tissue and cells in the peripheral area of the focused area. It means HIFU still has dangerousness to patients. The ultrasonic medical device in the present disclosure, which uses a low frequency and low intensity composite ultrasonic to destroy and emulsify a tumor tissue or cells, has many benefits. Because of the low operating temperature, the medical device may have delicate operation at the edge of the tumor tissue to clearly define the boundary of the tumor. The device may use to treat tumor with much more dangerousness and higher operation difficulty, and use to operations which need high technique such as cleaning the condensed plaques formed form neurodegenerative diseases in the brain. In various embodiments of the present disclosure, an ultrasonic conductor 390 may dispose between the ultrasonic transmitters 310B, 320B and a treatment body, which includes the affected area 350. To let the ultrasonic enter the treatment body through the ultrasonic conductor 390 to the affected area 350. The ultrasonic conductor 390 may include oil, water, emulsion, gel, or combinations thereof.

Referring to FIG. 5, FIG. 5 is a schematic diagram of an operation coordinate of an ultrasonic medical device according to various embodiments of the present disclosure. The ultrasonic transmitters may be operated by hands or controlled by a computer. Besides using the XYZ coordinate, which sets the origin in the affected area,the medical device can also use rΦ φ coordinate, in which r denotes a distance between the ultrasonic transmitter to the affected area, Φ denotes an angle between the ultrasonic transmitter and the Z coordinate, and φ denotes an horizontal angle between the ultrasonic transmitter and the x coordinate. Showing that the device may operate in a three dimensional space, and may adjust the directions and angles which the ultrasonics emit.

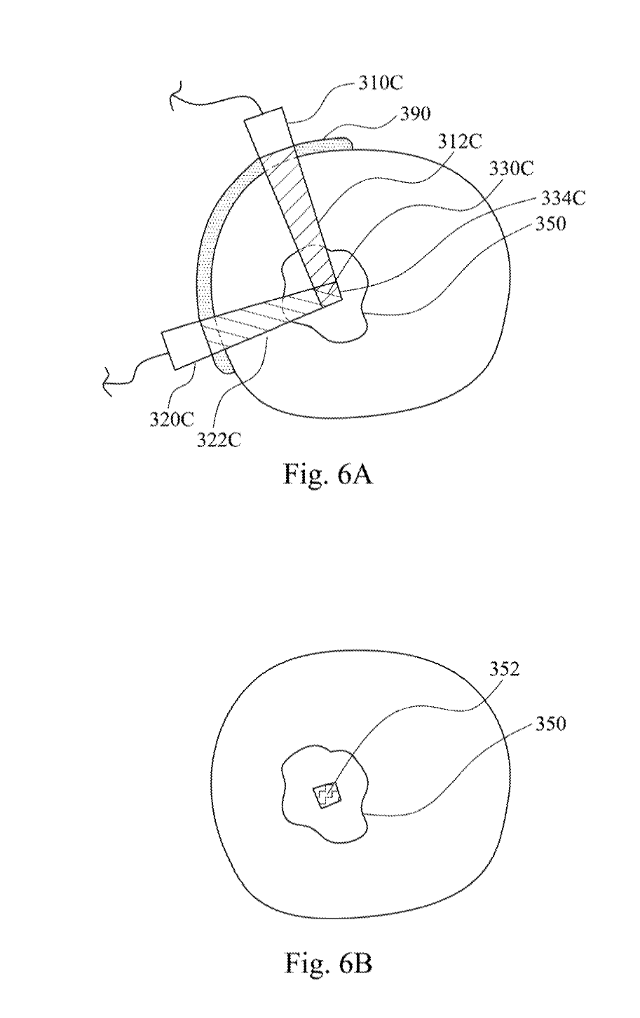

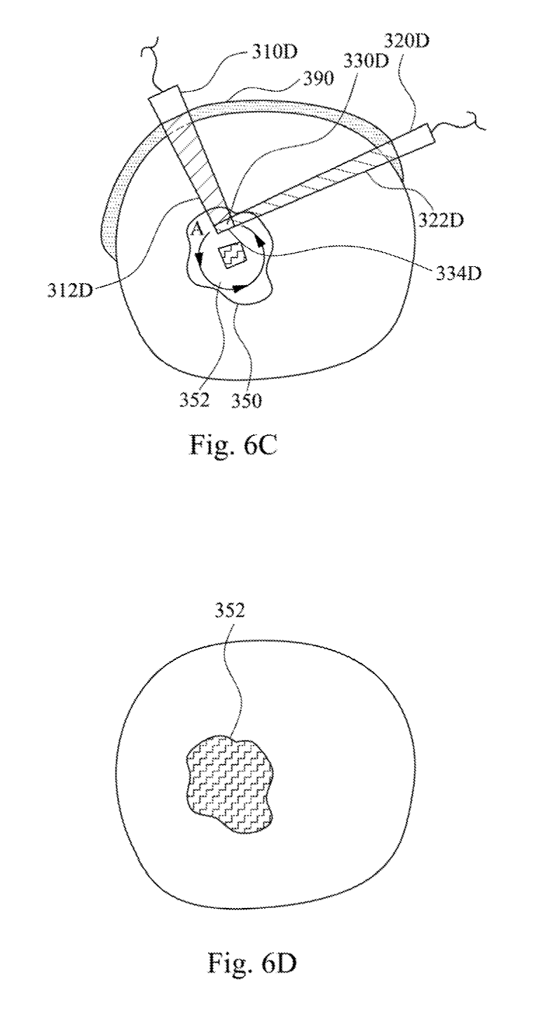

Referring to FIGS. 6A to 6D, FIGS. 6A to 6D are schematic diagrams of operation of ultrasonic medical device according to various embodiments of the present disclosure. Referring to FIG. 6A, in the depicted embodiment, the affected area 350 is a tumor. The treatment to the tumor is to completely destroy its tissue. In the beginning, the ultrasonic transmitters with larger area 310C, 320C are chosen. The first ultrasonic transmitter 310C emits the first ultrasonic 312C. The second ultrasonic transmitter 320C emits the second ultrasonic 322C. The ultrasonics 312C, 322C may enter the treatment body through the ultrasonic conductor 390, which is disposed between the ultrasonic transmitters 310C, 320C and the treatment body, to the affected area 350. A medical treatment area 334C is formed in the affected area 350 by intersecting the pathways of the two ultrasonics 312C, 322C. And the first composite ultrasonic 330C may be formed in the medical treatment area 334C. The first operation formed a larger medical treatment area, making the first composite ultrasonic 330C destroy most part of tissue and cells in the affected area. During the operation, the location of the ultrasonic transmitters 310C, 320C may be adjusted to destroy more cells and tissue in the affected area.

Referring to FIG. 6B, FIG. 6B is a schematic diagram of the affected area 350 after the treatment depicted in FIG. 6A. The tumor tissue in the medical treatment area, where the first ultrasonic 312C and the second ultrasonic 322C intersect, is destroyed and emulsify by the first composite ultrasonic, as the destroyed affected area 352 in the figure. Now only the boundary portions of the affected area 350, which are adjacent to normal tissue, are still tumor cells.

Referring to FIG. 6C, FIG. 6C depicts a schematic diagram of the second stage of the tumor tissue destroying method. The ultrasonic transmitters 310D, 320D with smaller cross section area may be chosen to treat the delicate part like the boundary of the affected area 350. The first ultrasonic transmitter 310D emits the first ultrasonic 312D. The second ultrasonic transmitter 320D emits the second ultrasonic 322D. The ultrasonics 312D, 322D may enter the treatment body through the ultrasonic conductor 390, which is disposed between the ultrasonic transmitters 310D, 320D and the treatment body, to the affected area 350. A medical treatment area 334D is formed in the affected area 350 by intersecting the pathways of the two ultrasonics 312D, 322D. And the first composite ultrasonic 330D may be formed in the medical treatment area 334D This stage of operation forms a smaller medical treatment area 334D. The location of the intersecting pathways of the two ultrasonics 312D, 322D emitted from the ultrasonic transmitters 310D, 320D may be moved around the boundary of the affected area 350, for example, circle around with direction A, to destroy the tumor cells and tissue not yet be destroyed in the operation of FIG. 6A in the affected area. The operation may repeat or use a ultrasonic transmitter with even a smaller cross section area to completely destroy the tumor tissue.

Referring to FIG. 6D, FIG. 6D shows a schematic diagram of the destroyed affected area 352. The former affected area 350 in FIG. 6A may completely be emulsified and transformed to the spoiled affected area 352. The tumor tissue and cells in the destroyed affected area 352 are all spoiled, also reaching the aim of tumor treatment without harming any cells or tissue out of the destroyed affected area 352. The emulsified tissue may be absorbed by human body naturally, or expelled out of body by a tube. The ultrasonic medical device according to various embodiments of the present disclosure may finely define the affected area or the tumor boundary in a delicate way due to mainly the ultrasonic mechanical effect.

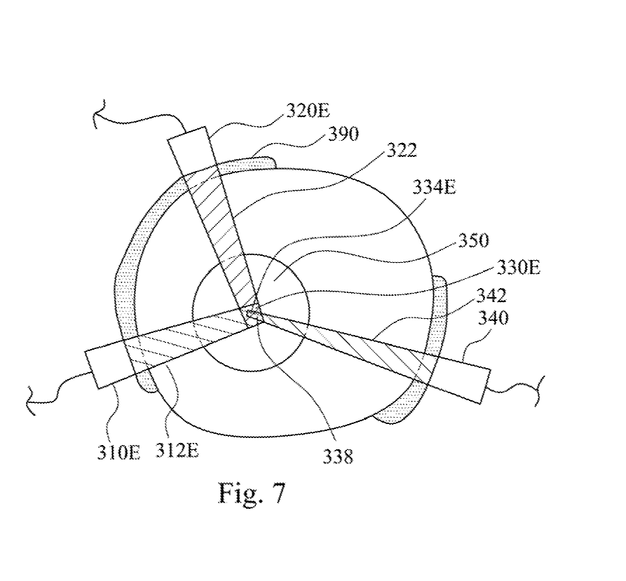

Referring to FIG. 7, FIG. 7 is a schematic diagram of an ultrasonic medical device according to various embodiments of the present disclosure. As depicted in the FIG. 7, the ultrasonic medical device includes a first ultrasonic transmitter 310E, a second ultrasonic transmitter 320E and a third ultrasonic transmitter 340. The first ultrasonic transmitter 310E emits a first ultrasonic 312E with frequency f1, intensity E1 and area A1. The second ultrasonic transmitter 320E emits a second ultrasonic 322E with frequency f2, intensity E2 and area A2. The third ultrasonic transmitter 340 emits a third ultrasonic 342 with frequency f4, intensity E4 and area A4. The area of the ultrasonic transmitters A1, A2 may be adjusted depending on the applying situation, the areas may also adjust continuously. In order to avoid the affected area is hindered by a bone, the third ultrasonic transmitter 340 may emit a third ultrasonic 342 from the opposite side. The pathways of the ultrasonics 312E, 322E may intersect for at least a portion to form a medical treatment area. The pathway of the third ultrasonic 342 may intersect at least a portion with the medical treatment area at the intersect area 334E. The medical treatment area is disposed in the affected area 350. In the mean while, the first and second ultrasonics 312E, 322E formed a first composite ultrasonic 330E with frequency f3, and f1, f2 are both larger than f3. The third ultrasonic 342 may interact with the first and second ultrasonics 312E, 322E, separately to generate a second composite ultrasonic with frequency f5, in which f1, f4 are both larger than f5, and a third composite ultrasonic with frequency f6, in which f2, f4 are both larger than f6. The first, second and third ultrasonics may together form at most three composite ultrasonics with different frequency f3, f5, and f6, which means three beats with different frequency in some embodiments. A fourth composite ultrasonic 338 is defined as a composite ultrasonic which is formed by the three ultrasonics 312E, 322E, 342 in the intersect area 334E including beats with at least one frequency. Thus the fourth composite ultrasonic may have stronger destructive power to accelerate the tumor tissue emulsifying process. In various embodiments of the present disclosure, a beam of the third ultrasonic 342 may be more convergent, making the intersect area 334 E, which includes the fourth composite ultrasonic 338 having higher destructive power including three different beat frequency f3, f5, f6, smaller than the area where the first composite ultrasonic 330E formed. In various embodiments of the present disclosure, using a plurality of ultrasonics with similar frequency to form a plurality of low frequency composite ultrasonics in their common intersecting area, having stronger destructive power to the tissue in the affected area, and may also decrease the temperature rising in the tissue. Further, the operation method may eliminate the difficulty to aim the ultrasonic transmitters 310E, 320E, and 340. Because the third ultrasonic 342 with narrower convergent beam may aim and emit into the place easily to form the intersect area 334E in the affected area 350, the operation may be faster and with much convenience. In various embodiments of the present disclosure, the first composite ultrasonic 330E has frequency about 40 kHz but with intensity only about 7 W/cm². Because the intensity is not enough to destroy the tumor tissue, the third ultrasonic 342 with proper frequency and intensity needs to be chosen to interact with the first and second ultrasonics 312E, 322E. To form the fourth composite ultrasonic 338 in the intersect area 334E having average intensity larger than 10 W/cm², and the frequency of the fourth composite ultrasonic 338 is in a range from about 20 to about 60 kHz, to reach the intensity that can destroy and emulsify the tumor tissue and cells. In operation, the position and the emitting direction of the third ultrasonic transmitter 340 and the convergence of the third ultrasonic 342 may adjust to emulsify the tissue where has the first composite ultrasonic 330E. Then by changing the intersect place of the three ultrasonics 312E, 342, 322E may scan and destroy the tissue in the affected area 350 completely. Besides, the frequency f4, intensity E4 and area A4 of the third ultrasonic 342, which is emitted by the third ultrasonic transmitter 340, may be adjusted. Because the intensity of the ultrasonics may be adjusted, the area A4 of the third ultrasonic transmitter 340 may be chosen larger, equal to, or smaller than the area A1, A2 of the first and second ultrasonic transmitters 310E, 320E depending on the operation convenience. In various embodiments of the present disclosure, the first ultrasonic 312E, second ultrasonic 322E, and the third ultrasonic 342 are all pulsed ultrasonics. In various embodiments of the present disclosure, an ultrasonic conductor 390 may dispose between the ultrasonic transmitters 310E, 320E, 340 and the treatment body. To let the ultrasonics enter the treatment body through the ultrasonic conductor 390 to the affected area 350. The ultrasonic conductor 390 may include oil, water, emulsion, gel, or combinations thereof.

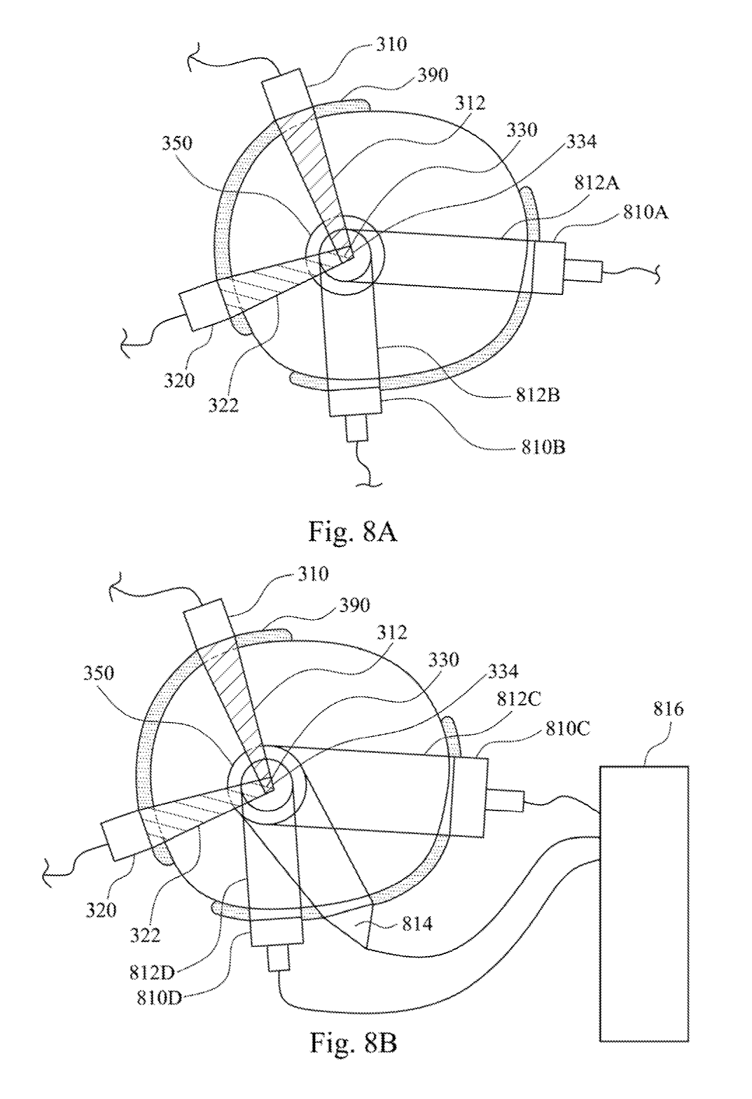

Referring to FIGS. 8A to 11B, in various embodiments disclosed in FIGS. 8A to 11B, the ultrasonic medical device includes a cooling device. As mentioned in the disclosure principle, part of the ultrasonic energy transformed to generate heat effect when forming the composite ultrasonic. Therefore, the cooling device may decrease the temperature in the medical treatment area and its peripheral, to eliminate the effect of the heat generation.

In contrast, the traditional HIFU which mainly depends on heating generation has the ultrasonic intensity about 1,000 W/cm² to 10,000 W/cm² at the focused area, and may burn the tissue and cells in the peripheral area of the sir focused area. It means HIFU still has dangerousness to patients. The principle of the medical device in the disclosure is based on the mechanical effect, therefore eliminating heat will not affect the operation of the ultrasonic medical device. On the contrary, the HIFU which is focusing on heat effect to burn the tissue may not cool the affected area, protein may denature at 54° C. and be spoiled. When the ultrasonic intensity is at 1,000 W/cm², the temperature of tissue may rise form 37° C. to about 50° C. Therefore, if the tissue temperature is decreased, the heat generated by the ultrasonic in the focus point may be eliminated. When the bulk tissue temperature around the focus point drops considerably, an internal energy and thermal fluctuation in the tissue may also decrease largely, so the heat dissipation ability of the tissue to against with the heat generated by the ultrasonics may be dramatically increasing in a nonlinear way. So the more temperature of the tissue around the focus point drops, the more heat may be eliminated, making the device much safer and also further expanding the frequency range of the first and second ultrasonics. In various embodiments of the present disclosure, the temperature of the medical treatment area is about 54° C. to about 0° C. For example, when the temperature of the medical treatment area is decreased to 5-8° C. The tissue at the focus area of the ultrasonic needs intensity over 10,000 W/cm² to make part of the ultrasonic energy to be absorbed to raise the temperature from 5° C. to about 54° C. So largely lowering the tissue temperature may promote heat dissipation ability of the tissue, also may effectively eliminate the heat generated by the ultrasonic in the focused area. In operation, one may use pulsed ultrasonics and a automatic temperature control system combined with a surgery control system to monitor and control the temperature of the medical treatment area during the operation. The surgery control system may automatically control the pulse intensity, pulse duration time, and pulse frequency of the pulsed ultrasonic to maintain the temperature in the medical treatment area in a safety range. Another benefit to combine the automatic temperature control system and the surgery control system as an integrated operation control system is that a security process may be developed. If the temperature of the medical treatment area increased up to 45° C. to 47° C. or even 50° C., in which the temperature upper limit of the medical treatment area is set to 50° C., the integrated operation control system may suspend the operation, waiting for tens of seconds or minutes to let the tissue cooled, then keep operating the above mentioned surgery. From an aspect of tissue cooling principle, the convergence and focus way may amplify the intensity of the ultrasonics in part of the area. However, the total average power of the ultrasonics emitting into the body is less than 1 W, so the total heat energy transferred from the ultrasonic energy absorbed by the tissue is not high, easily to be absorbed quickly by the bulk tissue in a low temperature environment, which can be seen as a low temperature reservoir. Also, adjusting the duty factor (DF) and the intensity of the pulsed ultrasonic may enhance the dissipation efficiency of the tissue, let the heat generated in a part area may be quickly and effectively absorbed by the surrounded tissue. In various embodiments of the present disclosure, the temperature of the medical treatment area after cooling is in a range from about 37° C. to about 0° C., the temperature in the medical treatment area is used to assist the above-mentioned automatic surgery. And the temperature of the medical treatment area may be adjusted depending on the operation but may not freeze the tissue in the area. In various embodiments of the present disclosure, the temperature in the medical treatment area is in a range from about 0° C. to about 54° C. In various embodiments of the present disclosure, the temperature in the medical treatment area is in a range from about 0° C. to about 50° C. In various embodiments of the present disclosure, the temperature in the medical treatment area is in a range from about 0° C. to about 45° C.

Referring to FIGS. 8A and 8B, FIG. 8A is a schematic diagram of an ultrasonic medical device according to various embodiments of the present disclosure. As depicted in FIG. 8A, the cooling device is an ultrasonic waveform elimination device. The ultrasonic waveform elimination device includes at least one phase-oppositing ultrasonic transmitter 810A. The phase-oppositing ultrasonic transmitter 810A emits at least one phase-oppositing ultrasonic 812A to the medical treatment area 334 and its peripheral. Using the principle of destructive interference to offset the heat amplitude in the above mentioned tissue to avoid the heat generated in the medical treatment area 334 injures the medical treatment area 334 and its peripheral. As shown in the figure, the phase-oppositing ultrasonic transmitter 810A is used to emit the phase-oppositing ultrasonic 812A to the medical treatment area 334 and its peripheral. In operation, another phase-oppositing ultrasonic transmitter 810B may be included, together with phase-oppositing ultrasonic transmitter 810A to emit phase-oppositing ultrasonic 812A, 812B separately on the same area. Or use a plurality of phase-oppositing ultrasonic transmitters to emit phase-oppositing ultrasonics in different angle to the medical treatment area 334 and its peripheral, to eliminate the heat amplitudes generated in the medical treatment area 334 and its peripheral. The frequency and intensity of the phase-oppositing ultrasonics are not in the range of the first and second ultrasonics 312, 322, therefore may not influence the effect of the first and second ultrasonics 312, 322. The phase-oppositing ultrasonic here means an ultrasonic having an out-of-phase waveform in relation with the heat amplitude, in order to eliminate the heat amplitude. Further, the heat generated in the medical treatment area, the same with the heat generated are may be largely eliminated, and a non-eliminated heat amplitude diffusing out of the medical treatment area to its peripheral can then be eliminated. Therefore, emitting the phase-oppositing ultrasonics may eliminate the heat generated in the heat generating area. The heat may not keep diffusing out to injure other peripheral tissue.

Referring to FIG. 8B, in FIG. 8B, an ultrasonic waveform elimination device as depicted in FIG. 8A further includes at least one ultrasonic sensor 814 and an ultrasonic analysis system 816. The ultrasonic sensor 814, phase-oppositing ultrasonic transmitters 810C, 810D are all connecting to the ultrasonic analysis system 816. As depicted in the figure, the phase-oppositing ultrasonic transmitters 810C, 810D may use different emitting area or adjusting the convergence or divergence area to offset heat amplitudes in different areas. Being assisted with the ultrasonic sensor 814 to sense the heat amplitude in the medical treatment area and its peripheral. Use the ultrasonic analysis system 816 to analyze the frequency, amplitude and phase angle of the heat amplitude, then emitting the phase-oppositing ultrasonics 812C, 812D, which are emitted by the phase-oppositing ultrasonic transmitters, and may eliminate the heat amplitudes in the tissue. Therefore, to eliminate temperature rising in the medical treatment area and its peripheral. Because the heat amplitude in the tissue may have spatial distribution, in some embodiments, one may use more than one phase-oppositing ultrasonic transmitters to emit phase-oppositing ultrasonics in different angles and directions. In various embodiments of the present disclosure, an ultrasonic conductor 390 may dispose between the phase-oppositing ultrasonic transmitters 810C, 810D and the treatment body. To let the ultrasonic enter the treatment body through the ultrasonic conductor 390 to the affected area 350. The ultrasonic conductor 390 may include oil, water, emulsion, gel, or combinations thereof.

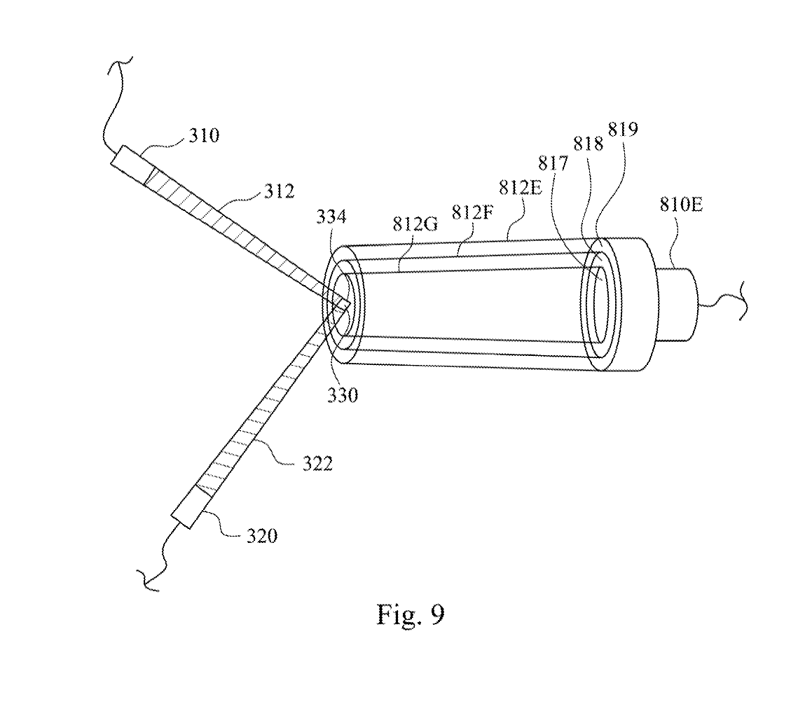

Referring to FIG. 9, FIG. 9 is a schematic diagram of a phase-oppositing ultrasonic transmitter of an ultrasonic medical device according to various embodiments of the present disclosure. As depicted in FIG. 9, a phase-oppositing ultrasonic transmitter 810E includes a plurality of concentric ring-shaped ultrasonic emitting surfaces 818, 819, a circle area 817 in the central of the emitting surface may also be an ultrasonic emitting surface. If the circle area 817 in the central of the emitting surface can emit ultrasonic, the area exposed to the phase-oppositing ultrasonic emitted by the phase-oppositing ultrasonic transmitter 810E is circle shape, where the exposed area includes the medical treatment area 334. If the circle area 817 in the central of the emitting surface cannot emit ultrasonic, the area exposed to the phase-oppositing ultrasonic emitted by the phase-oppositing ultrasonic transmitter 810E is ring shape. The phase-oppositing ultrasonics only eliminate the heat amplitude around the medical treatment area 334, the medical treatment area itself is not included. In various embodiments of the present disclosure, the circle area 817 in the central of the emitting surface may settle an ultrasonic sensor to sense the heat amplitude change in the medical treatment area 334 and its peripheral. Let the circle area 817 in the central of the emitting surface and the concentric ring-shaped ultrasonic emitting surfaces 818, 819 may emit the phase-oppositing ultrasonics, 812G, 812F, 812E with different waveforms to eliminate the heat amplitudes, which change with time and space in the medical treatment area and its peripheral. And the emitting surfaces 817, 818, 819 may emit different phase-oppositing ultrasonics to the medical treatment area and its peripheral from the small ring to the large ring sequentially or in the same time. In various embodiments of the present disclosure, the shape of the ultrasonic emitting surface of the phase-oppositing ultrasonic transmitter is circle, ring, polygon and combinations thereof.

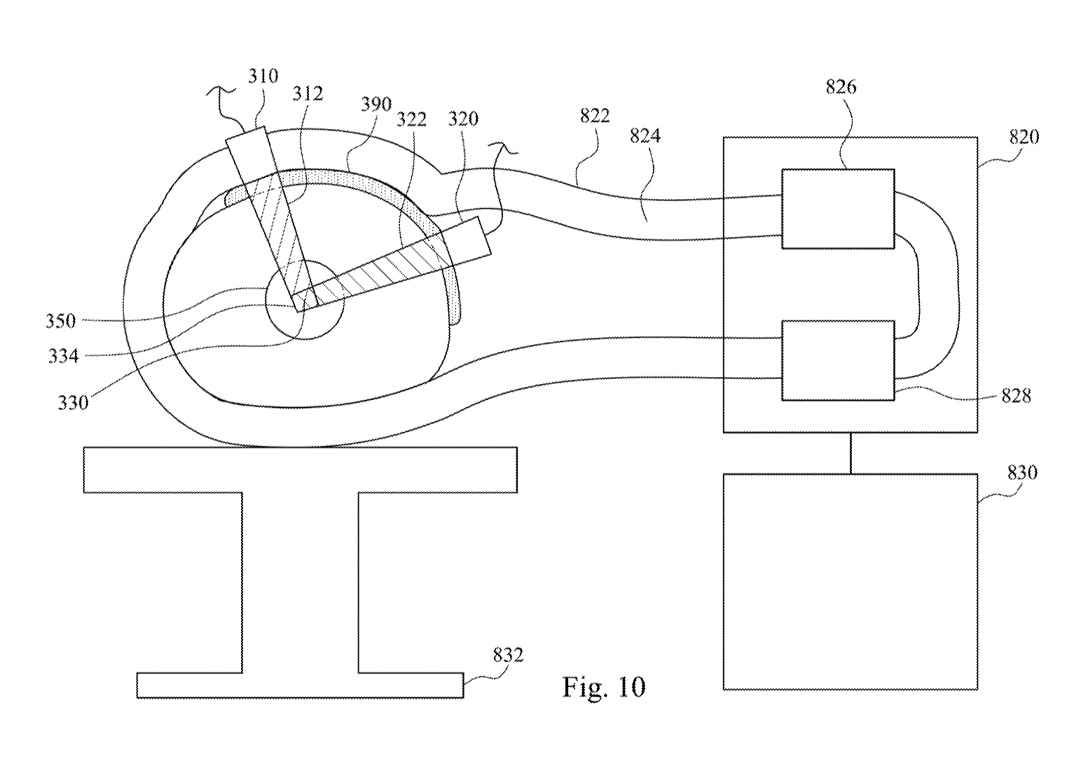

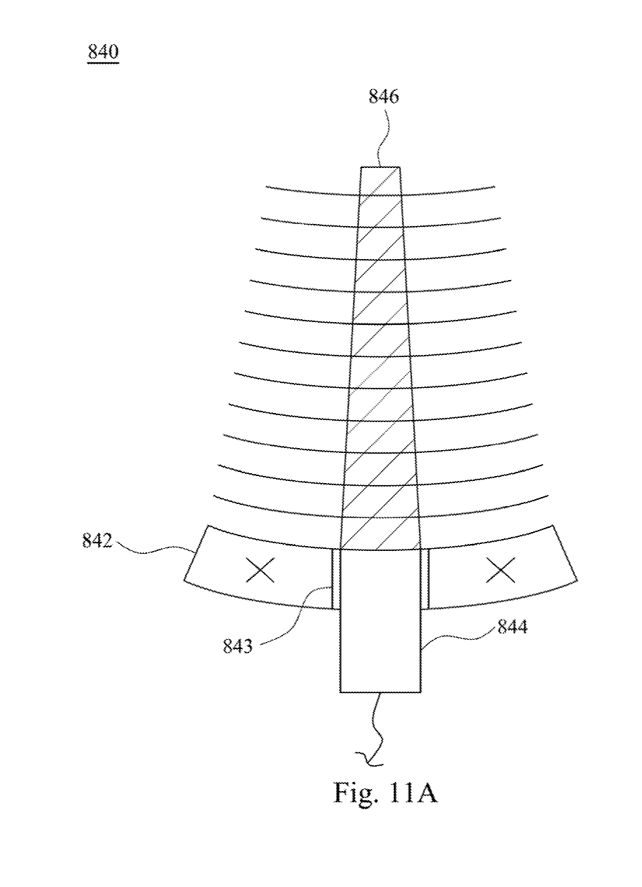

Referring to FIG. 10, FIG. 10 is a schematic diagram of a low temperature circulating cooling device of an ultrasonic medical device according to various embodiments of the present disclosure. As shown in the figure, the cooling device is the low temperature circulating cooling device. The low temperature circulating cooling device 820 includes a circulation system 822, which includes a coolant 824 flowing in the circulation system 822. A power device 826 is mounted in the circulation system 822, and a heat sink 828 is also mounted in the circulation system 822. As shown is the figure, the low temperature circulating cooling device 820 may be used together with an operating table 832 for the operating convenience. The low temperature circulating cooling device 820 uses the circulation system 822, which includes the coolant 824, surrounding the affected area 350 and part of its peripheral to lower the temperature of the tissue in the affected area and its peripheral. And using the power device 826 to propel the coolant 824 flowing to absorb heat, then use the heat sink 828 to cool the coolant 824, which has absorbed heat. In various embodiments of the present disclosure, the circulation system 822 may cover only one side of the peripheral of the affected area, not limited to surrounding all around the peripheral of the affected area. In various embodiments of the present disclosure, the coolant 824 may be cold water, antifreezer, or refrigerant; the power device 826 may be a motor; and the heat sink 828 may be a fan or a heat exchanger. In various embodiments of the present disclosure, the ultrasonic medical device further includes a temperature sensor system, which can detect the temperature of the affected area. The temperature sensor system may combine with an automatic control system to form an automatic temperature control system. The automatic temperature control system connects the cooling device and controls the temperature of the cooling device. In various embodiments of the present disclosure, the cooling device is a thermoelectric cooling device, which can directly contact and be fixed, for example, by a belt, on the affected area or its peripheral. The thermoelectric cooling device uses the thermoelectric effect of a semiconductor, and includes a thermoelectric cooling object, which can refrigerate, to lower the temperature in the affected area 350 and its peripheral. The thermoelectric cooling object may connect with a temperature adjusting system to sense and control the temperature in the affected area 350 and its peripheral. In various embodiments of the present disclosure, the cooling device further includes a local low temperature cooling kit, which can decrease the temperature in the medical treatment area. In various embodiments of the present disclosure, the local low temperature cooling kit includes a container, which have a capacity space and an endothermic substance, which is placed in the capacity space of the container. The container may dispose around the affected area to lower the temperature in the affected area, and may also be disposed on or under the operation table to cooling part of the operation table, which corresponds to the affected area, to lower the temperature of the affected area indirectly. The local low temperature cooling kit may touch the affected area or its peripheral and be fixed by a fixture to lower the temperature of the affected area and medical treatment area and its peripheral. In various embodiments of the present disclosure, the container may include flexible material, for example, a bag, disposed around the medical treatment area, and may be fixed around the affected area by a fixture. In various embodiments of the present disclosure, the container is a hard container, for example a tank or a bucket, disposed on or under the operation table, to cooling part of the operation table, which corresponds to the affected area, to lower the temperature of the affected area and its peripheral indirectly. The container may also have a special shape, which may closely contact the affected area or its peripheral. For example, a container with an arc contact surface may closely contact the abdomen, let the endothermic substance inside the container lowering the temperature of the affected area through the container. The container may have a large specific heat material to accelerate the cooling effect. In various embodiments of the present disclosure, the endothermic substance may be an ice, refrigerant, freezing mixture, antifreezer or other substance having endothermal effect.

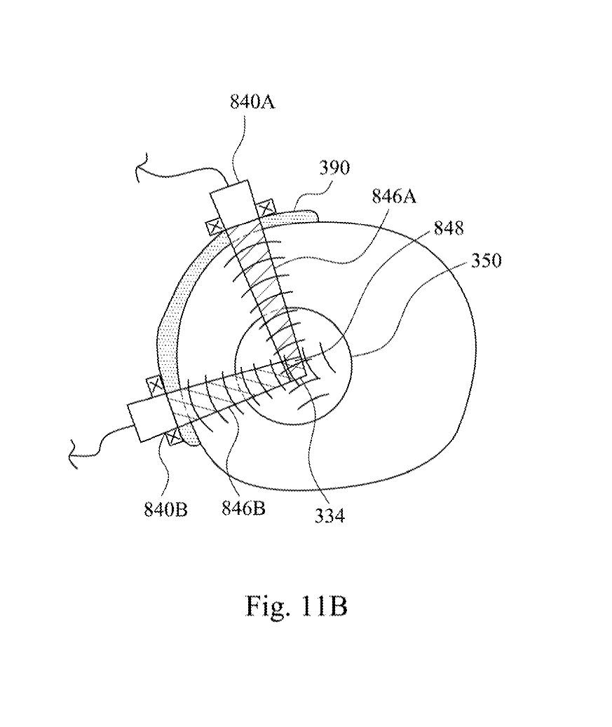

Referring to FIGS. 11A and 11B, FIG. 11A is a schematic diagram of a low temperature ultrasonic transmitter of an ultrasonic medical device according to various embodiments of the present disclosure. As depicted in the FIG. 11A, the cooling device may be a cooling attachment 842. The cooling attachment 842 sets around the ultrasonic transmitter 844 to form a low temperature ultrasonic transmitter 840. When operating the low temperature ultrasonic transmitter 840, an ultrasonic 846 may be emitted from the ultrasonic transmitter 844, and the cooling attachment 842 may provide cooling effect at the same time. In various embodiments of the present disclosure, the ultrasonic transmitter 844 and the cooling attachment 842 may be controlled separately, making the low temperature ultrasonic transmitter 840 only emitting the ultrasonics 846 or only cooling. In various embodiments of the present disclosure, the low temperature ultrasonic transmitter 840 further includes a partition 843 settled between the cooling attachment 842 and the ultrasonic transmitter 844 to prevent the low temperature affects the ultrasonic transmitter 844.

FIG. 11B is a schematic diagram of an ultrasonic medical device according to various embodiments of the present disclosure. FIG. 11B shows an operation of the low temperature ultrasonic transmitter 840 disclosed in FIG. 11A. The low temperature ultrasonic transmitters 840A, 840B emit a first ultrasonics 846A and a second ultrasonics 846B separately. The two ultrasonics 846A, 846B transfer into the affected area 350 through a conductor 390, intersecting and forming a medical treatment area 334 in the affected area 350 and lowering the temperature in the affected area 350 at the same time. A first composite ultrasonic 848 may be formed in the medical treatment area. The first composite ultrasonic 848 has enough intensity and right frequency to resonate with the tissue in the medical treatment area 334 and destroy them. In the meanwhile, the frequency of the ultrasonics 846A, 846B may be in a range from about 80 kHz to about 20 MHz. The intensity of the ultrasonics 846A, 846B may be in a range from about 1 mW/cm² to about 10 W/cm².

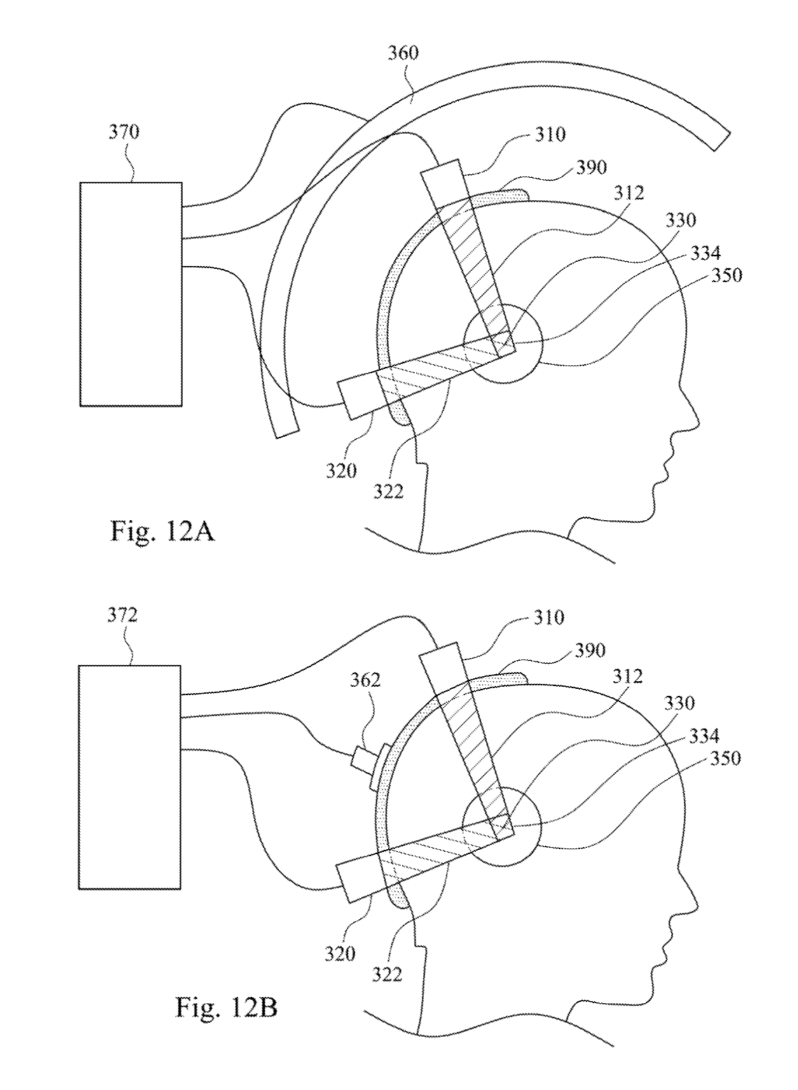

Referring to FIG. 12A, FIG. 12A is a schematic diagram of an ultrasonic medical device according to various embodiments of the present disclosure. In various embodiments of the present disclosure, the ultrasonic medical device may include a magnetic resonance imaging (MRI) 360 to assist defining the location of the affected area. The MRI 360 and an ultrasonic transmitter positioning and operation control system may be combined in an integrated operation control system 370. The integrated operation control system 370 may control and adjust the locations of the ultrasonic transmitters 310, 320, the emitting directions of the ultrasonics 312, 322, and the frequencies and intensities of the ultrasonics 312, 322 emitted by the ultrasonic transmitters 310, 320, and may monitor the images of the affected area in the same time. The integrated operation control system 370 may include an automatic temperature control system to automate the whole operation control, and also may monitor the images and temperature of the affected area. Therefore, enhancing the accuracy of the ultrasonic medical device.

Referring to FIG. 12B, FIG. 12B is a schematic diagram of an ultrasonic medical device according to various embodiments of the present disclosure. In various embodiments of the present disclosure, the ultrasonic medical device may include an ultrasonic tomography 362 to assist defining the location of the affected area 350. The ultrasonic tomography 362 and an ultrasonic transmitter positioning and operation control system may be combined in an integrated operation control system 372. The integrated operation control system 372 may control and adjust the locations of the ultrasonic transmitters 310, 320, the emitting directions of the ultrasonics 312, 322, and the frequencies and intensities of the ultrasonics 312, 322 emitted by the ultrasonic transmitters 310, 320, and may monitor the images of the affected area in the same time. The integrated operation control system 372 may include an automatic temperature control system to automate the whole operation control, and also may monitor the images and temperature of the affected area. Therefore, enhancing the accuracy of the ultrasonic medical device,

In FIGS. 3 to 12B, the disclosed embodiments are using the ultrasonic device to treat tumor. Using the composite ultrasonics with proper frequency and enough intensity to resonate with the tissue in the medical treatment area. The tissue and cells in the medical treatment area may be destroyed by resonance to reach an aim of destroying tumor without harming other tissue. At this point, the composite ultrasonic intensity in the medical treatment area is larger than 10 W/cm², or may be larger than 15, 20, even 25 W/cm² and with frequency between about 20-60 kHz to destroy tumor or fat. In various embodiments of the present disclosure, the frequency of the composite ultrasonics may be adjusted to about 50 to 80 kHz, and setting the medical treatment area into a fat tissue. The composite ultrasonics in this frequency range may resonate with fat tissue, having a fat-dissolving effect. In various embodiments of the present disclosure, the frequency of the composite ultrasonics may be adjusted to about 150 to 200 kHz, or using the composite ultrasonics with intensity about 10 to 20 W/cm². At this time, the frequency and intensity may not destroy cells, tissue or fat, but may still vibrate the body tissue to promote the blood circulation to increase the physical health. Therefore, the present disclosure is not only limited in tumor treatment, the disclosed ultrasonic medical device may have different application by adjusting the frequency and intensity of the ultrasonics.

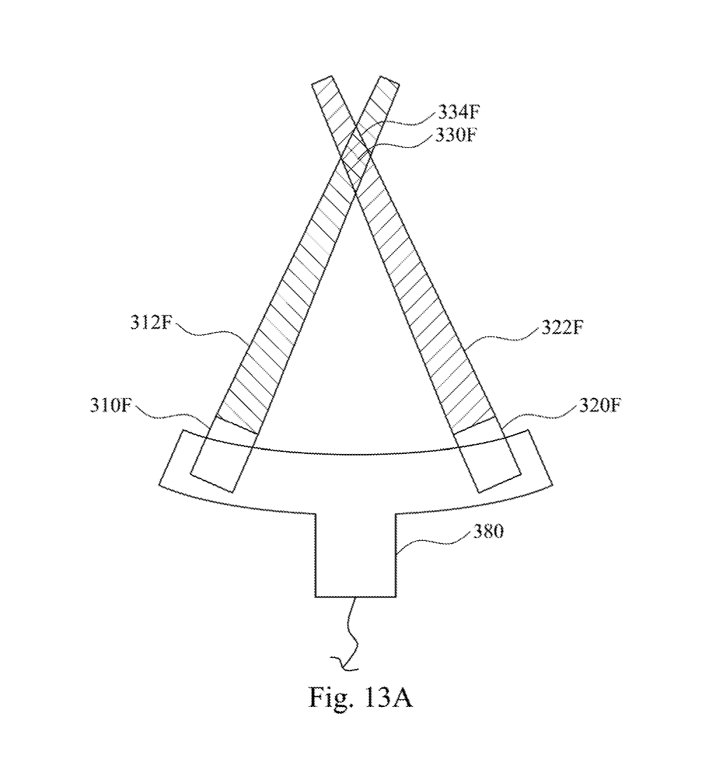

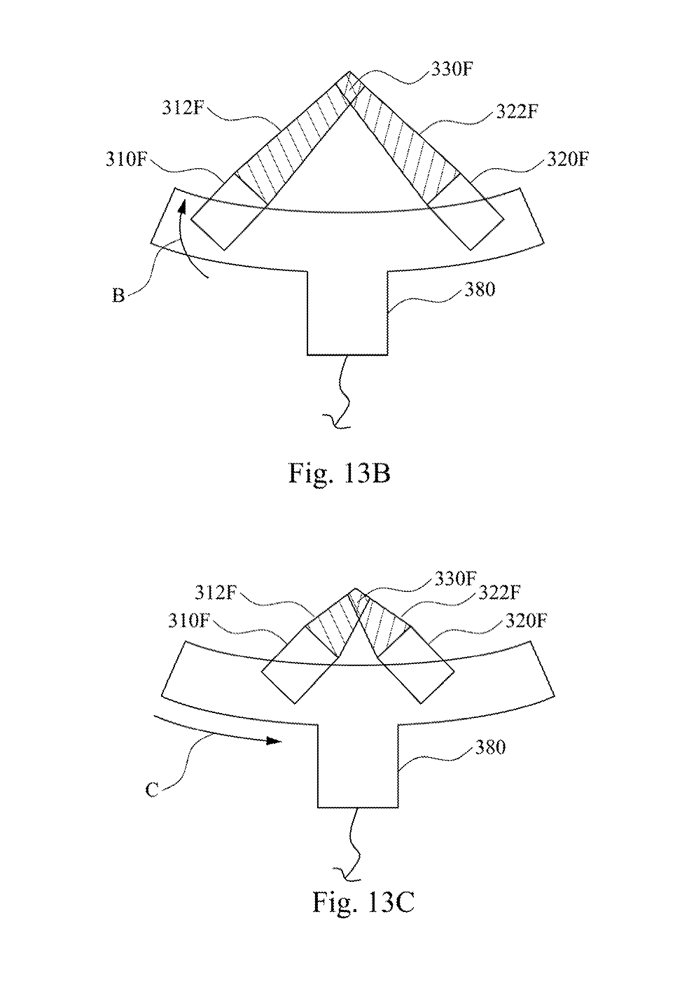

Referring to FIG. 13A, FIG. 13A is a schematic diagram of a composite probe of an ultrasonic medical device according to various embodiments of the present disclosure. As shown in the figure, two ultrasonic transmitters 310F, 320F are mounted on the composite probe 380. The first ultrasonic transmitter 310F emits the first ultrasonics 312F. The second ultrasonic transmitter 320F emits the second ultrasonics 322F. A medical treatment area 334F is formed in the affected area 350 by intersecting at least part of the pathways of the two ultrasonics 312F, 322F. And the first composite ultrasonic 330F may form in the medical treatment area 334F. The composite probe 380 may be operated by one hand. The composite probe 380 may treat skin disorders or dissolve fat in a close distance by setting the medical treatment area 334F near a surface, for example, body surface or skin.

Referring to FIG. 13B, FIG. 13B is a schematic diagram of a composite probe of an ultrasonic medical device according to various embodiments of the present disclosure. The operating method of the composite probe 380 is the same with the embodiments depicted in FIG. 13A. An emitting angle of the ultrasonic transmitters 310F, 320F may be adjusted, as a rotation direction B in the figure. Further, the emitting angles of the ultrasonic transmitters 310F, 320F may be adjusted to change the area where the first composite ultrasonic sir 330F is formed

Referring to FIG. 13C, FIG. 13C is a schematic diagram of a composite probe of an ultrasonic medical device according to various embodiments of the present disclosure. The operating method of the composite probe 380 is the same with the embodiments depicted in FIG. 13A. A distance between the is ultrasonic transmitters 310F, 320F and the convergence of the two ultrasonics 312F, 322F may be adjusted, as an ultrasonic transmitter moving direction C in the figure. Further, the moving of the ultrasonic transmitters 310F, 320F may change the distance between the area where the first composite ultrasonic 330F is formed and the surface.

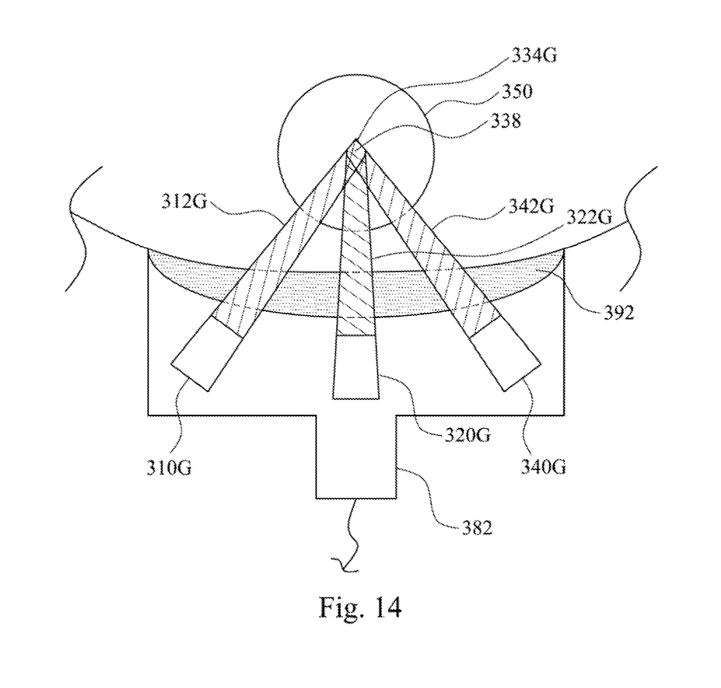

Referring to FIG. 14, FIG. 14 is a schematic diagram of a composite probe of an ultrasonic medical device according to various embodiments of the present disclosure. As depicted in the figure, three ultrasonic transmitters are mounted on the composite probe 382. A water bag 392 may dispose between the composite probe 382 and a treatment body, which includes the affected area 350, to let the ultrasonic enter the treatment body through the water bag 392 to the affected area 350. The first ultrasonic transmitter 310G emits the first ultrasonic 312G. The second ultrasonic transmitter 320G emits the second ultrasonic 322G. The third ultrasonic transmitter 340G emits the third ultrasonic 342G. A medical treatment area 334G is formed in the affected area 350 by intersecting at least part of the pathways of the three ultrasonics 312G, 322G, 342G. And the fourth composite ultrasonic 338 may form in the medical treatment area 334G. The emitting angle and the relative distance of the ultrasonic transmitters 310G, 320G, and 340G may be adjusted. The composite probe 382 may be operated by one hand, and may adjust where the fourth composite ultrasonic 338 is formed. The composite probe 382 has an emitting surface, which is a curved surface. The curved surface of the composite probe 382 may help adjusting the area, which the fourth composite ultrasonic 338 is formed, close to the surface. Besides, the ultrasonics 312G, 322G, 342G may be convergent beams. The composite probe 382 with the curved emitting surface may treat skin disorders, tumor near a body surface, remove freckle, or dissolve fat in a close distance by setting the medical treatment area 334G near a surface, for example, body surface or skin.

In various embodiments of the present disclosure, an ultrasonic temperature controlling method is provided, which applies for eliminating a heat generating from a heat generation area. The method includes sensing the first heat amplitude generating from the heat generation area and its peripheral, and analyzing the waveform of the first heat amplitude; and emitting a first phase-oppositing ultrasonic having an out-of-phase waveform in relation with the first heat amplitude to the heat generation area and its peripheral. The ultrasonic temperature controlling method is also applying the principle that the high frequency ultrasonic may not harm human tissue. The two out-of-phase ultrasonics may generate the destructive interference, one may analyze the heat amplitude in the heat generation area and its peripheral, and emit a first phase-oppositing ultrasonic having an out-of-phase waveform in relation with the heat amplitude to cancel the heat amplitude. Making the heat generated in the heat generation area may be largely eliminated. And the heat not yet be completely eliminated may diffuse to the peripheral of heat generation area, and the formed heat amplitude may then be eliminated. In various embodiments of the present disclosure, the above-mentioned ultrasonic temperature controlling method may be repeated. Keep sensing the second heat amplitude generating from the heat generation area and its peripheral, and analyzing the waveform of the second heat amplitude. In various embodiments of the present disclosure, the second heat amplitude is the residue of the first heat amplitude. Then emitting a second phase-oppositing ultrasonic having an out-of-phase waveform in relation with the second heat amplitude to the heat generation area and its peripheral for canceling the second heat amplitude. Therefore, emitting the phase-oppositing ultrasonic may eliminate the heat generated in the heat generation area. The heat may not further diffuse out to the peripheral of the heat generation area. The heat amplitudes in the tissue have various kinds of waveforms, temporal, and spatial distributions. In various embodiments of the present disclosure, a plurality of phase-oppositing ultrasonics with different frequency and different amplitude may be emitted in the same time to the heat generation area and its peripheral. In various embodiments of the present disclosure, the plurality of phase-oppositing ultrasonics may be emitted to the different part of the heat generation area and its peripheral to eliminate the heat amplitude in the tissue.

The method provides a non-harmful, using the ultrasonic wave property to eliminate the heat generated in the heat generation area to eliminate the heat diffusion. The method may also operate in a long time, and may apply in any situation needed to largely eliminate the heat and the heat diffusion. In various embodiments of the present disclosure, one may refer to the embodiments depicted in FIGS. 8A, 8B, where the device uses the principle of the ultrasonic waves which may generate destructive interference in the ultrasonic medical device to eliminate the heat injury in the heat generation area, i.e. the medical treatment area.

In various embodiments of the present disclosure, the ultrasonic medical device which is non-invasive and non-harmful has no radiation injury and no side effects of chemical treatments, which may define tumor boundary precisely, decrease device size largely, lower the device cost, and decrease the dangerousness of tumor treatment. The ultrasonic medical device may also apply in fat-dissolving, medical cosmetology, and blood circulation improving. In various embodiments of the present disclosure, a cooling device may include to expand the frequency and intensity range of the ultrasonics which may use to enhance the safety of the ultrasonic medical device.

In various embodiments of the present disclosure, an ultrasonic temperature controlling method is provided. By using the ultrasonic wave property to reach the aim of largely eliminating the heat generated in the heat generating area. The method may also apply in the ultrasonic medical device in the present disclosure to lower the temperature of the medical treatment area, making a better performance for the ultrasonic medical device disclosed herein.

Although the present invention has been described in considerable detail with reference to certain embodiments thereof, other embodiments are possible. Therefore, the spirit and scope of the appended claims should not be limited to the description of the embodiments contained herein.

It will be apparent to those skilled in the art that various modifications and variations can be made to the structure of the present invention without departing from the scope or spirit of the invention. In view of the foregoing, it is intended that the present invention cover modifications and variations of this invention provided they fall within the scope of the following claims.

What is claimed is:

1. An ultrasonic medical device, comprising:
a first ultrasonic transmitter, emitting a first ultrasonic with frequency f1; and
second ultrasonic transmitter, emitting a second ultrasonic with frequency f2;
wherein the first and the second ultrasonics are both convergent ultrasonics, pathways of these ultrasonics overlap with at least a portion of each other in a medical treatment area, forming a first composite ultrasonic with frequency f3 in the medical treatment area, both f1 and f2 are larger than f3, and the first composite ultrasonic has an average intensity larger than 10 W/cm².

2. The medical device of claim 1, further comprising a third ultrasonic transmitter, emitting a third ultrasonic, wherein the third ultrasonic is a convergent ultrasonic, the pathway of the third ultrasonic overlaps at least a portion of the medical treatment area, forming a fourth composite ultrasonic, together with the first and second ultrasonics in the medical treatment area, and the fourth composite ultrasonic has an average intensity larger than 10 W/cm².

3. The medical device of claim 1, wherein the convergent ultrasonics are focused ultrasonics.

4. The medical device of claim 1, further comprising a cooling device.

5. The medical device of claim 4, wherein the cooling device is selected from the group consisting of an ultrasonic waveform elimination device, a low temperature circulating cooling device, a thermoelectric cooling device, a local low temperature cooling kit and combinations thereof.

6. The medical device of claim 5, wherein the ultrasonic waveform elimination device comprises at least one phase-oppositing ultrasonic transmitter, emitting at least one phase-oppositing ultrasonic to the medical treatment area and its peripheral.

7. The medical device of claim 6, wherein the ultrasonic waveform elimination device further comprises at least one ultrasonic sensor, the ultrasonic sensor is used to sense a heat amplitude in the medical treatment area and its peripheral.

8. The medical device of claim 7, wherein the ultrasonic waveform elimination device further comprises an ultrasonic analysis system, connecting with the ultrasonic sensor and the phase-oppositing ultrasonic transmitter.

9. The medical device of claim 6, wherein a shape of the emitting surface of the phase-oppositing ultrasonic transmitter is circle, ring, polygon, and combinations thereof.

10. The medical device of claim 6, wherein the shape of the emitting surface of the phase-oppositing ultrasonic transmitter is a plurality of concentric rings, and the concentric rings emit different phase-oppositing ultrasonics to the medical treatment area and its peripheral from the small ring to the large ring simultaneously or sequentially.

11. The medical device of claim 5, wherein the low temperature circulating cooling device comprising:
a circulation system, comprising a coolant flowing in the circulation system;
a power device, mounted in the circulation system; and
a heat sink, mounted in the circulation system.

12. The medical device of claim 5, wherein the thermoelectric cooling device comprising:
a thermoelectric cooling object; and
a temperature adjusting system, connecting with the thermoelectric cooling object.

13. The medical device of claim 5, wherein the local low temperature cooling kit comprising:
a container, having a capacity space; and
an endothermic substance, placed in the capacity space of the container.

14. The medical device of claim 13, wherein the container is setting on or under an operating table.

15. The medical device of claim 4, further comprising an automatic temperature control system, comprising:
an automatic control system, connecting with the cooling device; and
a temperature sensor system, connecting with the automatic control system;
wherein the automatic temperature control system controls the temperature of the cooling device.

16. The medical device of claim 15, wherein the automatic temperature control system combines with an operation control system.

17. The medical device of claim 4, wherein the ultrasonic transmitter further comprises a cooling attachment, the cooling attachment sets around the ultrasonic transmitter to form a low temperature ultrasonic transmitter.

18. The medical device of claim 4, wherein a temperature in the medical treatment area is in a range from about 0° C. to about 37° C.

19. The medical device of claim 1, wherein a temperature in the medical treatment area is in a range from about 0° C. to about 54° C.

20. The medical device of claim 19, wherein the temperature in the medical treatment area is in a range from about 0° C. to about 50° C.

21. The medical device of claim 20, wherein the temperature in the medical treatment area is in a range from about 0° C. to about 45° C.

22. The medical device of claim 1, wherein an average intensity of the first composite ultrasonic is more than 15 W/cm².

23. The medical device of claim 22, wherein the average intensity of the first composite ultrasonic is more than 20 W/cm².

24. The medical device of claim 23, wherein the average intensity of the first composite ultrasonic is more than 25 W/cm².

25. The medical device of claim 1, wherein the medical treatment area is set in a tumor or a fat tissue.

26. The medical device of claim 25, wherein the first composite ultrasonic resonates with the tissue in the medical treatment area, the average resonance intensity is larger than 10 W/cm².

27. The medical device of claim 26, wherein the first composite ultrasonic resonates with the tissue in the medical treatment area, the average resonance intensity is larger than 15 W/cm².

28. The medical device of claim 27, wherein the average resonance intensity is larger than 20 W/cm².

29. The medical device of claim 28, wherein the average resonance intensity is larger than 25 W/cm².

30. The medical device of claim 1, wherein the frequencies of the ultrasonics are less than 10 times of the frequency of the first composite ultrasonic.

31. The medical device of claim 1, wherein the first composite ultrasonic is a beat and its frequency f3=|f1−f2|.

32. The medical device of claim 1, wherein the first and second ultrasonics are both pulsed ultrasonics.

33. The medical device of claim 32, wherein a pulse intensity, pulse duration time, and pulse frequency of the pulsed ultrasonics can be adjusted.

34. The medical device of claim 1, wherein the frequency of the first composite ultrasonic f3 is less than 200 kHz.

35. The medical device of claim 34, wherein the frequency of the first composite ultrasonic f3 is in a range from about 20 kHz to about 80 kHz.

36. The medical device of claim 35, wherein the frequency of the first composite ultrasonic f3 is in a range from about 20 kHz to about 60 kHz.

37. The medical device of claim 35, wherein the frequency of the first composite ultrasonic f3 is in a range from about 50 kHz to about 80 kHz.

38. The medical device of claim 34, wherein the frequency of the first composite ultrasonic f3 is in a range from about 150 kHz to about 200 kHz.

39. The medical device of claim 1, wherein a frequency, intensity, and the convergence of the ultrasonics emitted by the ultrasonic transmitter can be adjusted.

40. The medical device of claim 1, wherein the frequency of the ultrasonic emitted by the ultrasonic transmitter is in a range from about 80 kHz to about 20 MHz.

41. The medical device of claim 1, wherein the intensity of the ultrasonic emitted by the ultrasonic transmitter is in a range from about 1 mW/cm² to about 10 W/cm².

42. The medical device of claim 1, wherein the cross sectional area of the first ultrasonic is larger, equal to, or smaller than the cross sectional area of the second ultrasonic.

43. The medical device of claim 1, wherein the focal length of the ultrasonic emitted by the ultrasonic transmitter can be adjusted.

44. The medical device of claim 1, wherein the focus area of the ultrasonic emitted by the ultrasonic transmitter can be adjusted.

45. The medical device of claim 1, wherein the spatial relationships between the ultrasonic transmitters can be defined by a perpendicular relative angle Φ and a horizontal relative angle θ.

46. The medical device of claim 1, further comprising a magnetic resonance imaging or an ultrasonic tomography to define the affected area.

47. The medical device of claim 1, further comprising an integrated operation control system, the system can control and adjust the locations of the ultrasonic transmitters, the emitting directions of the ultrasonics, and the frequencies and intensities of the ultrasonics emitted by the ultrasonic transmitters.

48. The medical device of claim 1, further comprising a composite probe, the composite probe having an emitting surface, wherein the ultrasonic transmitters mount on the emitting surface of the composite probe, and the distance between the ultrasonic transmitters and the emitting angles of the ultrasonic transmitters on the composite probe can be adjusted.

49. The medical device of claim 48, wherein the emitting surface of the composite probe is a curved surface.

50. The medical device of claim 48, further comprising a water bag disposed between the composite probe and the medical treatment area.

51. An ultrasonic temperature controlling method for eliminating a heat generated from a heat generation area, comprising:
sensing the first heat amplitude generating from the heat generation area and its peripheral, and analyzing the waveform of the first heat amplitude; and
emitting a first phase-oppositing ultrasonic having an out-of-phase waveform in relation with the first heat amplitude to the heat generation area and its peripheral.

52. The temperature controlling method of claim 51, further comprising:
sensing the second heat amplitude generating from the heat generation area and its peripheral, and analyzing the waveform of the second heat amplitude, wherein the second heat amplitude is the residue of the first heat amplitude; and
emitting a second phase-oppositing ultrasonic having an out-of-phase waveform in relation with the second heat amplitude to the heat generation area and its peripheral.

53. The temperature controlling method of claim 51, wherein in emitting a first phase-oppositing ultrasonic having an out-of-phase waveform in relation with the first heat amplitude to the heat generation area and its peripheral comprising:
emitting a plurality of phase-oppositing ultrasonics with different frequencies to the heat generation area and its peripheral.

54. The temperature controlling method of claim 51, wherein in emitting a first phase-oppositing ultrasonic having an out-of-phase waveform in relation with the first heat amplitude to the heat generation area and its peripheral comprising:
emitting a plurality of phase-oppositing ultrasonics with different amplitudes to the heat generation area and its peripheral.

55. The temperature controlling method of claim 51, wherein in emitting a first phase-oppositing ultrasonic having an out-of-phase waveform in relation with the first heat amplitude to the heat generation area and its peripheral comprising:
emitting a plurality of phase-oppositing ultrasonics to the different parts of the heat generation area and its peripheral.
